(19) United States
(12) Patent Application Publication
(10) Pub. No.: US 2026/0204417 A1
(43) Pub. Date: Jul. 16, 2026

(54) METHODS AND SYSTEMS FOR AI-DRIVEN REFINEMENT OF MENTAL HEALTH DIAGNOSTICS AND PERSONALIZED TREATMENT

(71) Applicant: Neoptio Health Incorporated, Vancouver (CA)

(72) Inventor: Christopher David McNamara, Vancouver (CA)

(73) Assignee: Neoptio Health Incorporated, Vancouver (CA)

(21) Appl. No.: 19/564,195

(22) Filed: Mar. 12, 2026

Related U.S. Application Data
(63) Continuation of application No. PCT/IB2024/062255, filed on Dec. 5, 2024.
(60) Provisional application No. 63/606,574, filed on Dec. 5, 2023.

(51) Int. Cl. G16H 50/20

(52) U.S. Cl. CPC G16H 50/20

(57) ABSTRACT

The present disclosure provides a method of facilitating AI-driven refinement of mental health diagnostics and personalized treatment. The method may include transmitting a prompt data to a user device. Further, the method may include receiving a mental health data from the user device based on the prompt data. Further, the mental health data may be associated with a user. Further, the method may include identifying a demography data based on the mental health data. Further, the method may include determining a diagnostic schema data to provide diagnostic insights based on the demography data. Further, the method may include generating a pre-diagnosis and/or diagnosis report data to provide personalized insights based on the diagnostic schema data and the mental health data. Further, the method may include transmitting the pre-diagnosis and/or diagnosis report data to the user device.

### CROSS-REFERENCE TO RELATED APPLICATIONS

This application is a US bypass continuation application of the PCT application PCT/IB2024/062255, tiled “METHODS AND SYSTEMS FOR AI-DRIVEN REFINEMENT OF MENTAL HEALTH DIAGNOSTICS AND PERSONALIZED TREATMENT”, filed on December 5, 2024, which in turn claims benefit of the United States provisional patent application No. 63/606,574, titled “METHODS AND SYSTEMS FOR FACILITATING INTERACTIVE COLLECTION, ANALYSIS, AND DIGITAL THERAPEUTICS IN MENTAL HEALTH”, filed on 12/05/2023, each of which is incorporated by reference herein in its entirety.

### FIELD OF DISCLOSURE

The present invention relates generally to the field of data processing in healthcare. More specifically, the present invention pertains to methods and systems for AI-driven refinement of mental health diagnostics and personalized treatment.

### BACKGROUND

The field of digital data processing in healthcare is critically important to various industries, organizations, and individuals. Recent advancements in healthcare services, driven by digital technologies, have significantly optimized workflows, diagnostics, and treatments, benefiting patients, clinicians, and administrators alike.

Despite these advancements, existing digital mental healthcare services typically rely on standardized diagnostic frameworks such as the DSM or ICD, which lack the ability to analyze mental health conditions at a granular level. As a result, these frameworks do not provide personalized diagnostic approaches tailored to the unique characteristics of an individual’s mental health.

Furthermore, digital mental healthcare services that offer automated pre-diagnosis, diagnosis, and therapy are often limited by the unstructured nature of mental health data and inconsistencies in data formats, leading to inefficiencies and delays in processing.

Therefore, there is a need for innovative methods and systems for AI-driven refinement of mental health diagnostics and personalized treatment. Such systems would enable more granular, data-driven analysis and tailored therapeutic interventions for individual patients.

### SUMMARY OF DISCLOSURE

This summary is provided to introduce a selection of concepts in a simplified form that are further described below in the Detailed Description. This summary is not intended to identify key features or essential features of the claimed subject matter, nor is it intended to limit the claimed subject matter’s scope.

The present disclosure provides a method of facilitating AI-driven refinement of mental health diagnostics and personalized treatment. The method may include transmitting prompt data to a user device via a communication device, and receiving mental health data from the user device based on the prompt data. This mental health data may be associated with the user. The method may further include identifying demographic data based on the mental health data and using it to determine a diagnostic schema. Using a processing device, the method may generate a pre-diagnosis and/or diagnosis report based on both the diagnostic schema and the mental health data. This report may then be transmitted to the user device.

The present disclosure also provides a system for facilitating AI-driven refinement of mental health diagnostics and personalized treatment. The system includes a communication device configured to transmit prompt data to a user device, receive mental health data from the user device, and transmit a pre-diagnosis and/or diagnosis report back to the user device. Additionally, the system includes a processing device that identifies demographic data associated with the user, determines diagnostic schema data based on the demographic data, and generates the pre-diagnosis and/or diagnosis report accordingly.

Furthermore, the present disclosure describes a method of transforming mental health data into more granular, individual data points. The method may include analyzing this data and generating a hierarchical structure that enables more refined, personalized diagnostic conclusions using machine learning techniques. Each data point may be associated with a specific index, allowing access within the hierarchical structure. A rule engine is then employed to evaluate the data based on diagnostic schema rules, which may be dynamically refined over time. The system continually refines and personalizes diagnoses based on accumulated data, ultimately generating a report tailored to the individual's unique mental health profile.

### BRIEF DESCRIPTIONS OF DRAWINGS

The accompanying drawings, which are incorporated in and constitute a part of this disclosure, illustrate various embodiments of the present disclosure. The drawings contain representations of various trademarks and copyrights owned by the Applicants. In addition, the drawings may contain other marks owned by third parties and are being used for illustrative purposes only. All rights to various trademarks and copyrights represented herein, except those belonging to their respective owners, are vested in and the property of the applicants. The applicants retain and reserve all rights in their trademarks and copyrights included herein, and grant permission to reproduce the material only in connection with reproduction of the granted patent and for no other purpose.

Furthermore, the drawings may contain text or captions that may explain certain embodiments of the present disclosure. This text is included for illustrative, non-limiting, explanatory purposes of certain embodiments detailed in the present disclosure.

FIG. 1 is an illustration of an online platform 100 consistent with various embodiments of the present disclosure.

FIG. 2 is a block diagram of a computing device 200 for implementing the methods disclosed herein, in accordance with some embodiments.

FIG. 3 illustrates a flowchart of a method 300 for generating and providing personalized mental health diagnostics and treatment, in accordance with some embodiments.

FIG. 4 illustrates a flowchart of a method 400 for updating the diagnostic schema data using the processing device 704 as part of providing personalized mental health diagnostics, in accordance with some embodiments.

FIG. 5 illustrates a flowchart of a method 500 for generating therapy data using the processing device 704 as part of providing personalized mental health treatment, in accordance with some embodiments.

FIG. 6 illustrates a flowchart of a method 600 for determining alternative therapy data using the processing device 704, in accordance with some embodiments.

FIG. 7 illustrates a block diagram of a system 700 for providing personalized mental health diagnostics and treatment, in accordance with some embodiments.

FIG. 8 illustrates a flowchart of a method 800 for updating the domain machine learning model using the processing device 704, in accordance with some embodiments.

FIG. 9 illustrates a flowchart of a method 900 for determining the plurality of medical domain indicators using the processing device 704, in accordance with some embodiments.

FIG. 10 illustrates a flowchart of a method 1000 for generating progress report data using the processing device 704, in accordance with some embodiments.

FIG. 11 illustrates a flowchart of a method 1100 for generating a compliance parameter using the processing device 704, in accordance with some embodiments.

FIG. 12 illustrates a flowchart of a method 1200 for generating notification data using the processing device 704, in accordance with some embodiments.

FIG. 13 illustrates a flowchart of a method 1300 for generating helpline data using the processing device 704, in accordance with some embodiments.

FIG. 14 illustrates a flowchart of a method 1400 for receiving medical condition data from an external data source using the communication device 702, in accordance with some embodiments.

FIG. 15A illustrates a flowchart of a method 1500 for generating therapy report data using the processing device 704, in accordance with some embodiments.

FIG. 15B illustrates a continuation of the flowchart of method 1500 for generating therapy report data using the processing device 704, in accordance with some embodiments.

FIG. 16A illustrates a flowchart of a method 1600 for providing personalized mental health diagnostics and treatment, in accordance with some embodiments.

FIG. 16B illustrates a continuation of the flowchart of method 1600 for providing personalized mental health diagnostics and treatment, in accordance with some embodiments.

FIG. 17 illustrates a flowchart of a system providing a high-level view of components and their interactions, in accordance with some embodiments.

FIG. 18 illustrates a flowchart of proprietary diagnostic schema showcasing the structure and functionality of the diagnostic schema, illustrating the categorization, organization, and analysis of the data, in accordance with some embodiments.

FIG. 19 illustrates a diagram of the user interface, wherein user provides the input data using various methods like text, voice, video, and image, in accordance with some embodiments.

FIG. 20 illustrates a flowchart of specialized sub-modules for different medical specialties and processing and analyzing performed on relevant data, in accordance with some embodiments.

FIG. 21 illustrates a flowchart of the backend algorithm detailing steps to process data, generate a pre-diagnosis and diagnosis, and propose personalized digital therapeutics, in accordance with some embodiments.

FIG. 22 illustrates a flowchart of the personalized digital therapeutics module, detailing the generation and presentation of personalized digital therapeutics based on collected medical history and pre-diagnosis/diagnosis, in accordance with some embodiments.

FIG. 23 illustrates a flowchart of data privacy compliance system showing the data flow within the system with encryption and other security measures to comply with healthcare data privacy regulations, in accordance with some embodiments.

FIG. 24 illustrates a flowchart of external medical databases integration detailing the integration and interaction of external medical databases with the machine learning module and diagnostic schema, in accordance with some embodiments.

FIG. 25 illustrates a flowchart of continuous improvement mechanism illustrating the feedback loop from user feedback and system performance monitoring to the refinement of various system components, in accordance with some embodiments.

FIG. 26 illustrates a diagram of future extension provisions illustrating integration of new diagnosis ontology and additional data sources into the system in future, in accordance with some embodiments.

### DETAILED DESCRIPTION OF DISCLOSURE

As a preliminary matter, it will readily be understood by one having ordinary skill in the relevant art that the present disclosure has broad utility and application. As should be understood, any embodiment may incorporate only one or a plurality of the above-disclosed aspects of the disclosure and may further incorporate only one or a plurality of the above-disclosed features. Furthermore, any embodiment discussed and identified as being “preferred” is considered to be part of a best mode contemplated for carrying out the embodiments of the present disclosure. Other embodiments also may be discussed for additional illustrative purposes in providing a full and enabling disclosure. Moreover, many embodiments, such as adaptations, variations, modifications, and equivalent arrangements, will be implicitly disclosed by the embodiments described herein and fall within the scope of the present disclosure.

Accordingly, while embodiments are described herein in detail in relation to one or more embodiments, it is to be understood that this disclosure is illustrative and exemplary of the present disclosure, and are made merely for the purposes of providing a full and enabling disclosure. The detailed disclosure herein of one or more embodiments is not intended, nor is to be construed, to limit the scope of patent protection afforded in any claim of a patent issuing here from, which scope is to be defined by the claims and the equivalents thereof. It is not intended that the scope of patent protection be defined by reading into any claim limitation found herein and/or issuing here from that does not explicitly appear in the claim itself.

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the present disclosure. Accordingly, it is intended that the scope of patent protection is to be defined by the issued claim(s) rather than the description set forth herein.

Additionally, it is important to note that each term used herein refers to that which an ordinary artisan would understand such term to mean based on the contextual use of such term herein. To the extent that the meaning of a term used herein—as understood by the ordinary artisan based on the contextual use of such term—differs in any way from any particular dictionary definition of such term, it is intended that the meaning of the term as understood by the ordinary artisan should prevail.

Furthermore, it is important to note that, as used herein, “a” and “an” each generally denotes “at least one,” but does not exclude a plurality unless the contextual use dictates otherwise. When used herein to join a list of items, “or” denotes “at least one of the items,” but does not exclude a plurality of items of the list. Finally, when used herein to join a list of items, “and” denotes “all of the items of the list.”

The following detailed description refers to the accompanying drawings. Wherever possible, the same reference numbers are used in the drawings and the following description to refer to the same or similar elements. While many embodiments of the disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the disclosure. Instead, the proper scope of the disclosure is defined by the claims found herein and/or issuing here from. The present disclosure contains headers. It should be understood that these headers are used as references and are not to be construed as limiting upon the subjected matter disclosed under the header.

The present disclosure includes many aspects and features. Moreover, while many aspects and features relate to, and are described in the context of the disclosed use cases, embodiments of the present disclosure are not limited to use only in this context.

In general, the method disclosed herein may be performed by one or more computing devices. For example, in some embodiments, the method may be performed by a server computer in communication with one or more client devices over a communication network such as, for example, the Internet. In some other embodiments, the method may be performed by one or more of at least one server computer, at least one client device, at least one network device, at least one sensor and at least one actuator. Examples of the one or more client devices and/or the server computer may include, a desktop computer, a laptop computer, a tablet computer, a personal digital assistant, a portable electronic device, a wearable computer, a smart phone, an Internet of Things (IoT) device, a smart electrical appliance, a video game console, a rack server, a super-computer, a mainframe computer, mini-computer, micro-computer, a storage server, an application server (e.g. a mail server, a web server, a real-time communication server, an FTP server, a virtual server, a proxy server, a DNS server etc.), a quantum computer, and so on. Further, one or more client devices and/or the server computer may be configured for executing a software application such as, for example, but not limited to, an operating system (e.g. Windows, Mac OS, Unix, Linux, Android, etc.) in order to provide a user interface (e.g. GUI, touch-screen based interface, voice based interface, gesture based interface etc.) for use by the one or more users and/or a network interface for communicating with other devices over a communication network. Accordingly, the server computer may include a processing device configured for performing data processing tasks such as, for example, but not limited to, analyzing, identifying, determining, generating, transforming, calculating, computing, compressing, decompressing, encrypting, decrypting, scrambling, splitting, merging, interpolating, extrapolating, redacting, anonymizing, encoding and decoding. Further, the server computer may include a communication device configured for communicating with one or more external devices. The one or more external devices may include, for example, but are not limited to, a client device, a third party database, public database, a private database and so on.

Further, the communication device may be configured for communicating with the one or more external devices over one or more communication channels. Further, the one or more communication channels may include a wireless communication channel and/or a wired communication channel. Accordingly, the communication device may be configured for performing one or more of transmitting and receiving of information in electronic form. Further, the server computer may include a storage device configured for performing data storage and/or data retrieval operations. In general, the storage device may be configured for providing reliable storage of digital information. Accordingly, in some embodiments, the storage device may be based on technologies such as, but not limited to, data compression, data backup, data redundancy, deduplication, error correction, data finger-printing, role based access control, and so on.

Further, one or more steps of the method disclosed herein may be initiated, maintained, controlled and/or terminated based on a control input received from one or more devices operated by one or more users such as, for example, but not limited to, an end user, an admin, a service provider, a service consumer, an agent, a broker and a representative thereof. Further, the user as defined herein may refer to a human, an animal or an artificially intelligent being in any state of existence, unless stated otherwise, elsewhere in the present disclosure. Further, in some embodiments, the one or more users may be required to successfully perform authentication in order for the control input to be effective. In general, a user of the one or more users may perform authentication based on the possession of a secret human readable secret data (e.g. username, password, passphrase, PIN, secret question, secret answer etc.) and/or possession of a machine readable secret data (e.g. encryption key, decryption key, bar codes, etc.) and/or or possession of one or more embodied characteristics unique to the user (e.g. biometric variables such as, but not limited to, fingerprint, palm-print, voice characteristics, behavioral characteristics, facial features, iris pattern, heart rate variability, evoked potentials, brain waves, and so on) and/or possession of a unique device (e.g. a device with a unique physical and/or chemical and/or biological characteristic, a hardware device with a unique serial number, a network device with a unique IP/MAC address, a telephone with a unique phone number, a smartcard with an authentication token stored thereupon, etc.). Accordingly, the one or more steps of the method may include communicating (e.g. transmitting and/or receiving) with one or more sensor devices and/or one or more actuators in order to perform authentication. For example, the one or more steps may include receiving, using the communication device, the secret human readable data from an input device such as, for example, a keyboard, a keypad, a touch-screen, a microphone, a camera and so on. Likewise, the one or more steps may include receiving, using the communication device, the one or more embodied characteristics from one or more biometric sensors.

Further, one or more steps of the method may be automatically initiated, maintained and/or terminated based on one or more predefined conditions. In an instance, the one or more predefined conditions may be based on one or more contextual variables. In general, the one or more contextual variables may represent a condition relevant to the performance of the one or more steps of the method. The one or more contextual variables may include, for example, but are not limited to, location, time, identity of a user associated with a device (e.g. the server computer, a client device etc.) corresponding to the performance of the one or more steps, environmental variables (e.g. temperature, humidity, pressure, wind speed, lighting, sound, etc.) associated with a device corresponding to the performance of the one or more steps, physical state and/or physiological state and/or psychological state of the user, physical state (e.g. motion, direction of motion, orientation, speed, velocity, acceleration, trajectory, etc.) of the device corresponding to the performance of the one or more steps and/or semantic content of data associated with the one or more users. Accordingly, the one or more steps may include communicating with one or more sensors and/or one or more actuators associated with the one or more contextual variables. For example, the one or more sensors may include, but are not limited to, a timing device (e.g. a real-time clock), a location sensor (e.g. a GPS receiver, a GLONASS receiver, an indoor location sensor etc.), a biometric sensor (e.g. a fingerprint sensor), an environmental variable sensor (e.g. temperature sensor, humidity sensor, pressure sensor, etc.) and a device state sensor (e.g. a power sensor, a voltage/current sensor, a switch-state sensor, a usage sensor, etc. associated with the device corresponding to performance of the or more steps).

Further, the one or more steps of the method may be performed one or more number of times. Additionally, the one or more steps may be performed in any order other than as exemplarily disclosed herein, unless explicitly stated otherwise, elsewhere in the present disclosure. Further, two or more steps of the one or more steps may, in some embodiments, be simultaneously performed, at least in part. Further, in some embodiments, there may be one or more time gaps between performance of any two steps of the one or more steps.

Further, in some embodiments, the one or more predefined conditions may be specified by the one or more users. Accordingly, the one or more steps may include receiving, using the communication device, the one or more predefined conditions from one or more and devices operated by the one or more users. Further, the one or more predefined conditions may be stored in the storage device. Alternatively, and/or additionally, in some embodiments, the one or more predefined conditions may be automatically determined, using the processing device, based on historical data corresponding to performance of the one or more steps. For example, the historical data may be collected, using the storage device, from a plurality of instances of performance of the method. Such historical data may include performance actions (e.g. initiating, maintaining, interrupting, terminating, etc.) of the one or more steps and/or the one or more contextual variables associated therewith. Further, machine learning may be performed on the historical data in order to determine the one or more predefined conditions. For instance, machine learning on the historical data may determine a correlation between one or more contextual variables and performance of the one or more steps of the method. Accordingly, the one or more predefined conditions may be generated, using the processing device, based on the correlation.

Further, one or more steps of the method may be performed at one or more spatial locations. For instance, the method may be performed by a plurality of devices interconnected through a communication network. Accordingly, in an example, one or more steps of the method may be performed by a server computer. Similarly, one or more steps of the method may be performed by a client computer. Likewise, one or more steps of the method may be performed by an intermediate entity such as, for example, a proxy server. For instance, one or more steps of the method may be performed in a distributed fashion across the plurality of devices in order to meet one or more objectives. For example, one objective may be to provide load balancing between two or more devices. Another objective may be to restrict a location of one or more of an input data, an output data and any intermediate data corresponding to one or more steps of the method. For example, in a client-server environment, sensitive data corresponding to a user may not be allowed to be transmitted to the server computer. Accordingly, one or more steps of the method operating on the sensitive data and/or a derivative thereof may be performed at the client device.

### Overview

The present disclosure describes methods and systems for the interactive collection, analysis, and provision of digital therapeutics in mental health. Ensuring the secure handling and storage of sensitive medical history data, preliminary diagnoses, and proposed digital therapeutics is a significant concern, given the stringent healthcare data privacy regulations. Further, the disclosed system may be configured for interactive collection and analysis of medical history across diverse medical specialties, with a focus on mental health. It employs machine learning techniques and a proprietary diagnostic schema to generate personalized pre-diagnoses, diagnoses, and provide tailored digital therapeutics.

Further, the system may be designed to revolutionize the interactive collection and analysis of medical history, particularly emphasizing mental health. By employing a proprietary diagnostic schema dynamically refined through machine learning techniques based on accumulated medical history data, user feedback, and external medical databases, the system provides more personalized diagnoses and the provision of tailored digital therapeutics.

Key Components of the system may include:

Proprietary Diagnostic Schema: A unique framework that evolves over time, enhancing diagnostic accuracy and personalization of digital therapeutics.

Machine Learning Techniques: Employed to continually refine the diagnostic schema based on various data inputs, ensuring the system stays updated with emerging medical knowledge and individual patient profiles.

Cross-Specialty Interaction: Specialized sub-modules provided for different medical specialties, enabling a holistic approach to mental health care by integrating insights from other medical domains.

Interactive Data Collection: Utilizing a user-friendly interface to collect medical history data through various input methods, presenting a blend of standardized and proprietary questions tailored to diverse medical specialties.

Backend Algorithm: Processes responses to generate a pre-diagnosis and/or diagnosis for clinician review, and proposes personalized digital therapeutics based on the collected data and refined diagnostic schema.

The system aims to address the limitations of existing digital diagnostic and therapeutic tools by providing a more dynamic, personalized, and integrated approach to mental health care. Further, the system endeavors to significantly improve the diagnostic accuracy, treatment efficacy, and overall patient experience in the mental health domain.

The invention provides a comprehensive system and method for the interactive collection and analysis of medical history across various medical specialties, with a primary focus on mental health.

The core of the system includes a proprietary diagnostic schema that is dynamically refined over time through machine learning techniques. This schema provides more personalized diagnoses by adapting to accumulated medical history data, user feedback, and external medical databases.

Machine learning algorithms may be employed to continually refine the diagnostic schema, ensuring that the system evolves with emerging medical knowledge and individual patient profiles.

The system provides specialized sub-modules for different medical specialties including mental health, neurology, and psychiatry. Each sub-module is configured to process input types relevant to the respective medical specialty, enabling a holistic approach to mental health care. A user-friendly interface may be employed to collect medical history data through various input methods, presenting a blend of standardized and proprietary questions tailored to diverse medical specialties. A robust backend algorithm processes responses to generate a pre-diagnosis and/or diagnosis for clinician review. It also proposes personalized digital therapeutics based on the collected data and refined diagnostic schema.

Based on the collected medical history and refined diagnostic schema, personalized digital therapeutics are proposed to address individual patient needs, particularly in the domain of mental health. The system ensures that all medical history data, preliminary diagnoses, and proposed digital therapeutics are stored securely in compliance with healthcare data privacy regulations. Provisions are made for future extensions to incorporate unique diagnostic ontology and additional data sources, allowing the system to stay relevant and adaptive to evolving medical knowledge. The user interface is designed for ease of use and accessibility across diverse patient demographics, ensuring that the system is inclusive and user-friendly.

Further, the system employs a proprietary diagnostic schema that is dynamically refined over time through machine learning techniques based on accumulated medical history data, user feedback, and/or external medical databases, facilitating more personalized diagnoses and the provision of tailored digital therapeutics. The system is configured to collect medical history data through a user interface presenting a blend of standardized and proprietary question sets. Further, data input methods include text, voice, video, image, and provisions for future integration of haptic and biometric data. Further, a backend algorithm processes responses to generate a pre-diagnosis and/or diagnosis for clinician review. Further, the system comprises a provision for future extensions to incorporate unique diagnostic ontology and additional data sources. Further, personalized digital therapeutics are proposed based on the collected medical history and pre-diagnosis and/or diagnosis. Further, the system may include specialized sub-modules provided for different medical specialties including mental health, neurology, psychiatry, and others. Further, each specialized sub-module is configured to process input types relevant to the respective medical specialty. Further, the system may be configured for generating a pre-diagnosis and/or diagnosis based on interactive collection and analysis of medical history. Further, the medical history data is collected through a series of questions presented via a user-friendly interface. Further, data is collected through text, voice, video, image, and provisions for future integration of haptic and biometric data. Further, a backend algorithm processes responses to propose a pre-diagnosis and/or diagnosis for clinician review. Further, the system may include a provision for proposing personalized digital therapeutics based on the collected data. Further, the question sets are developed based on public health frameworks and proprietary criteria. Further, the system is designed for extension to include additional data sources and diagnostic ontology. Further, the system may include a non-transitory computer-readable medium comprising instructions for executing the generation of the pre-diagnosis and/or diagnosis. Further, the user interface is designed for ease of use and accessibility across diverse patient demographics. Further, the backend algorithm employs machine learning techniques to continually refine the proprietary diagnostic schema and personalized digital therapeutics recommendations based on accumulated medical history data, user feedback, and/or external medical databases. Further, the proposed digital therapeutics are personalized to individual patient profiles. Further, the medical history data, pre-diagnosis and/or diagnosis, and proposed digital therapeutics are stored securely in compliance with healthcare data privacy regulations.

The proprietary diagnostic schema at the core of the invention is designed to dynamically evolve and refine over time, providing a more personalized approach to mental health diagnostics and treatment based on individual patient data and feedback. Through the integration of machine learning techniques, the diagnostic schema continually refines itself based on accumulated medical history data, user feedback, and external medical databases. This dynamic nature ensures the system stays updated with emerging medical knowledge and individual patient profiles, unlike static algorithms employed by existing solutions. The system includes specialized sub-modules for different medical specialties, enabling a more holistic approach to mental health care by integrating insights from other medical domains. This cross-specialty interaction is often lacking in current digital health solutions. With a user-friendly interface and a variety of data input methods, the invention allows for a more interactive and comprehensive collection of medical history data. This contrasts with inflexible data collection methods employed by some existing tools. The robust backend algorithm processes responses to generate a pre-diagnosis and/or diagnosis for clinician review and proposes personalized digital therapeutics, facilitating an efficient and effective treatment planning process.

The system has robust data security measures ensuring all medical history data, preliminary diagnoses, and proposed digital therapeutics are stored securely in compliance with healthcare data privacy regulations. Provisions are made for future extensions to incorporate unique diagnostic ontology and additional data sources, allowing the invention to remain relevant and adaptive to evolving medical knowledge, which is a feature often lacking in existing solutions. Designed for ease of use and accessibility across diverse patient demographics, the user-friendly interface encourages user engagement and accurate data collection, addressing the problem of complex or non-intuitive interfaces in some existing tools. The continuous refinement of the diagnostic schema based on machine learning ensures that the system improves over time, enhancing diagnostic accuracy, treatment efficacy, and overall patient experience in the mental health domain. The system provides a more comprehensive, adaptive, and user-friendly solution for mental health care in the digital realm. The system has the potential to significantly improve the quality of mental health care delivery, making it a superior alternative to existing solutions in the market.

Further, in some embodiments, the emotion sensor may be a camera and/or voice recognition devices. Further, in some embodiments, the eye-tacking sensor may be a camera. Further, in some embodiments, the optical sensor may be a camera. Further, in some embodiments, the motion sensor may be a camera.

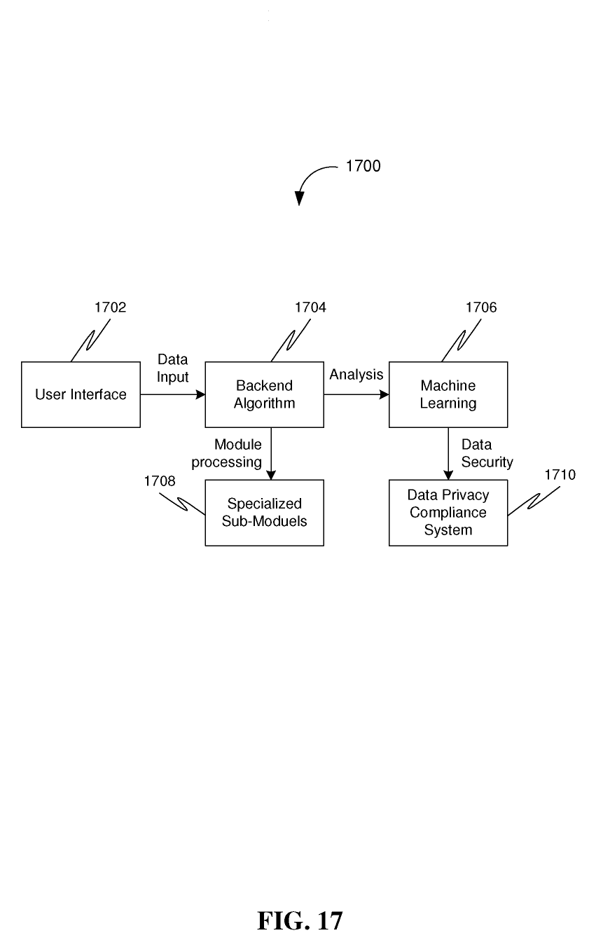

Further, referring to FIG. 17, the system overview 1700, in some embodiments, may comprise user interface 1702, backend algorithm 1704, machine learning model 1706, specialized sub-modules 1708, data privacy compliance system 1710. Further, the user interface 1702 may be used to transmit and receive data from the user in the form of text data, image data, voice data, video data, haptic data and/or biometric data. Further, the user interface 1702 may be communicatively coupled with the backend algorithm 1704 to transmit the data input and the backend algorithm may further perform processing and analyzing of the received data corresponding to specialized sub-modules 1708 to generate a pre-diagnosis and/or diagnosis for clinician review and personalized digital therapeutics based on the diagnostic outcome. Further the analyzed data may be transferred to a machine learning model 1706 that is communicatively coupled with the backend algorithm 1704. Further the machine learning model 1706 may provide data privacy to the analyzed data based on data privacy compliance system 1710.

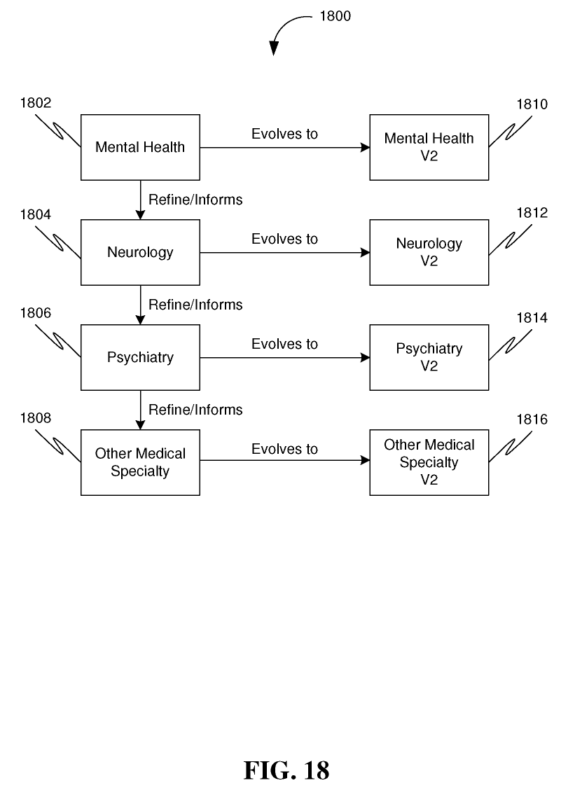

Further, referring to FIG. 18, the proprietary diagnostic schema 1800, in some embodiments, may comprise mental health 1802, mental health V21810, neurology 1804, neurology V21812, psychiatry 1806, psychiatry 1814, other medical specialty 1808, and other medical specialty V21816, to perform evolution of each medical specialty to the corresponding sub specialty. Additionally, each medical specialty may refine to the another medical specialty upon analysis.

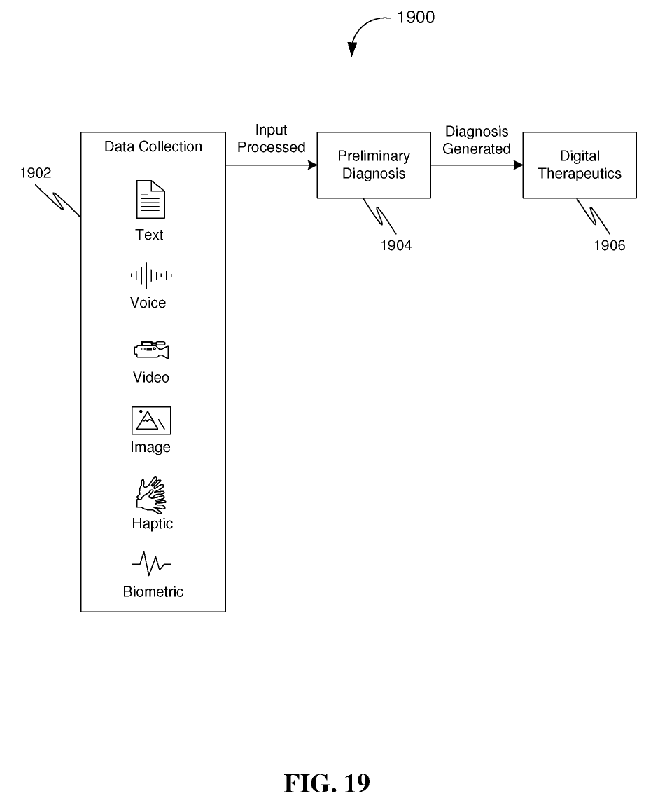

Further, referring to FIG. 19, in some embodiments, the user interface system 1900 may receive by data collection 1902, the input data in the form of text data, voice data, video data, image data, haptic data and biometric data. The input data may further undergo processing and analyzation to generate a pre-diagnosis and/or diagnosis 1904. The pre-diagnosis and/or diagnosis 1904 may undergo further analysis to generate digital therapeutics 1906.

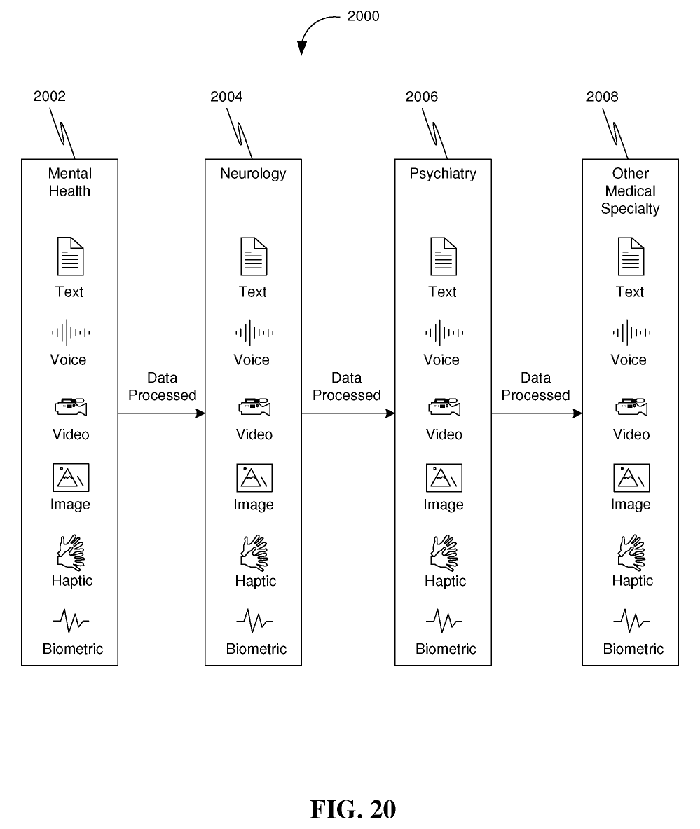

Further, referring to FIG. 20, in some embodiments, specialized sub-modules 2000 may comprise, the input data in the form of text data, voice data, video data, image data, haptic data and/or biometric data present in each medical specialty comprising mental health 2002, neurology 2004, psychiatry 2006 and other medical specialty 2008 may further be processed leading to evolution of each medical specialty data to another medical specialty data.

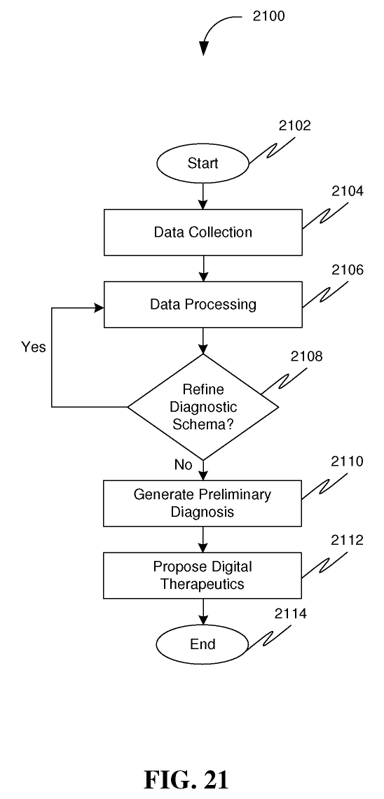

Further, referring to FIG. 21, in some embodiments, the backend algorithm 2100 may perform data collection 2104 from the user and performing data processing 2106. Further, the backend algorithm 2100 may check for the need for refining of the diagnostic schema 2108. Further, the backend algorithm 2100 may generate pre-diagnosis and/or diagnosis 2110 based on diagnostic schema 2108. Further, it may propose a digital therapeutics 2112 based on the pre-diagnosis and/or diagnosis 2110.

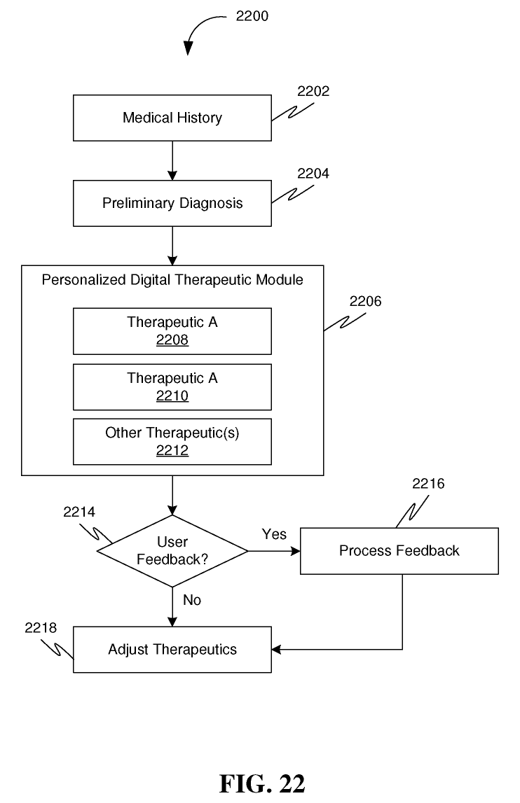

Further, referring to FIG. 22, in some embodiments, the personalized digital therapeutics module method 2200 may comprise a pre-diagnosis and/or diagnosis 2204 of medical history 2202 received from the user. Further, it may generate a personalized digital therapeutics module 2206 comprising several therapeutics, for instance, therapeutic A 2208, therapeutic B 2210, other therapeutics 2212 based on the pre-diagnosis and/or diagnosis 2204. Further, it may comprise a user feedback system 2214 configured to adjust therapeutics 2220 based on the processing of the user feedback 2216.

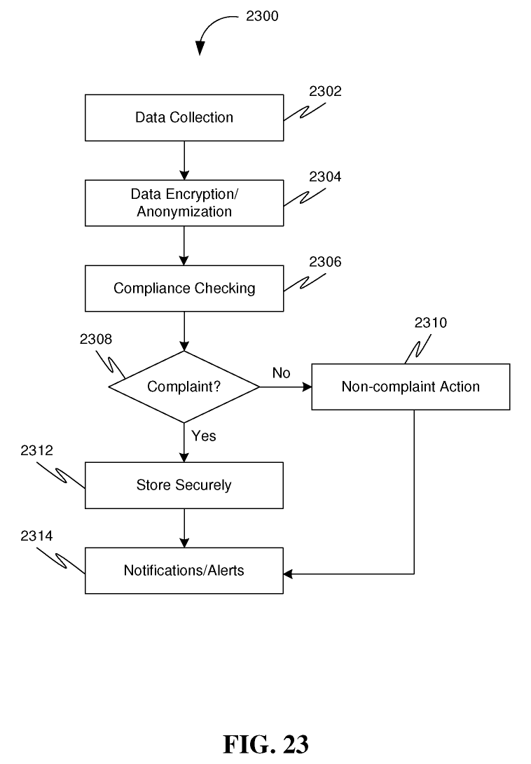

Further, referring to FIG. 23, the data privacy compliance system 2300, in some embodiments, may perform data collection 2302, data encryption 2304 and/or compliance checking 2306 of the encrypted data. Further, the compliance checking 2306 may include storing securely 2312 the data in case of compliancy 2308 and generate an alert 2314 in case of non-compliancy 2310.

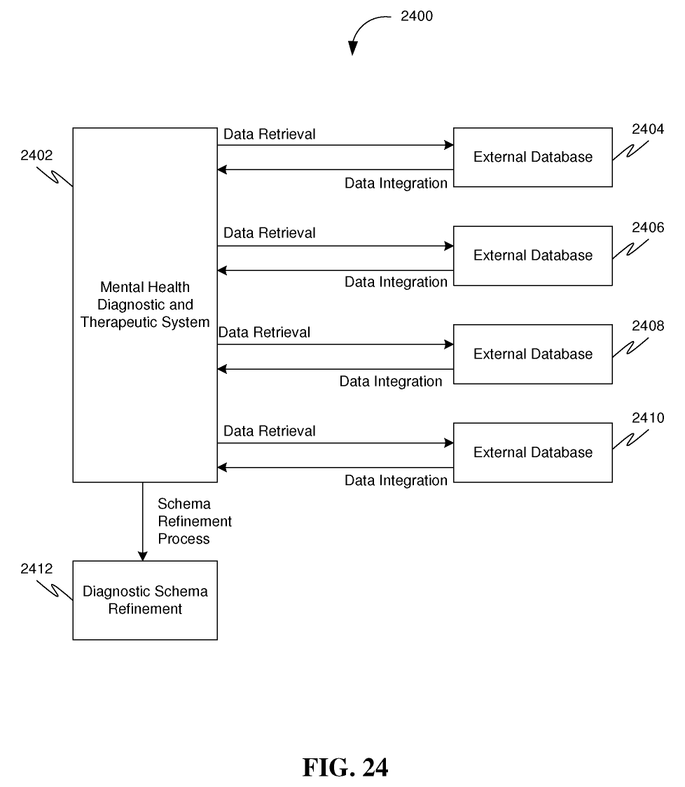

Further, referring to FIG. 24, in some embodiments, the external medical databases integration method 2400 may include integration of external medical databases 2404, 2406, 2408, 2410 with the mental health diagnostic and therapeutic system 2402 to retrieve data from the external databases. Further, it may include diagnostic schema refinement 2412 based on the data retrieved from the external medical database.

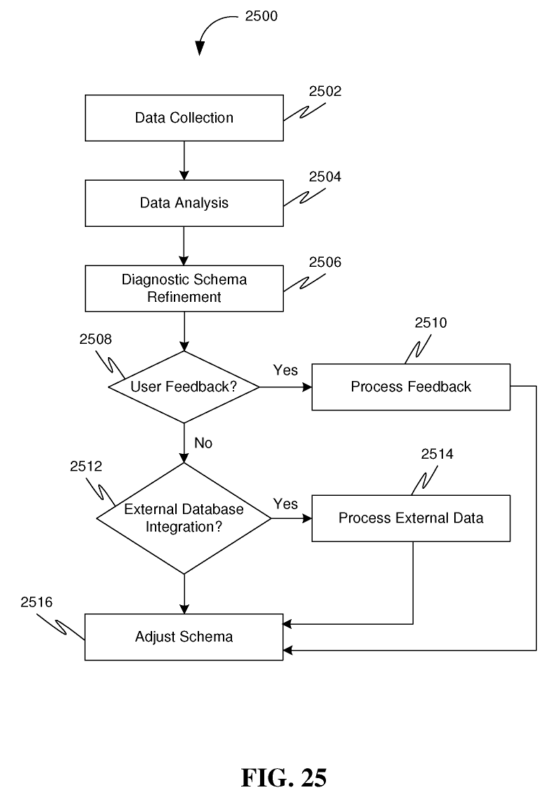

Further, referring to FIG. 25, in some embodiments, the continuous improvement mechanism 2500 may include continuous improvement mechanism performing data collection 2502, data analysis 2504 and diagnostic schema refinement 2506 based on the data analysis 2504. Further, the diagnostic schema may be adjusted based on the user feedback 2508 upon processing the feedback 2510. Further, it may integrate with external database 2512 and process the external data 2514 to adjust schema 2516. Further, it may adjust the diagnostic schema by integrating with external database upon receiving a user feedback.

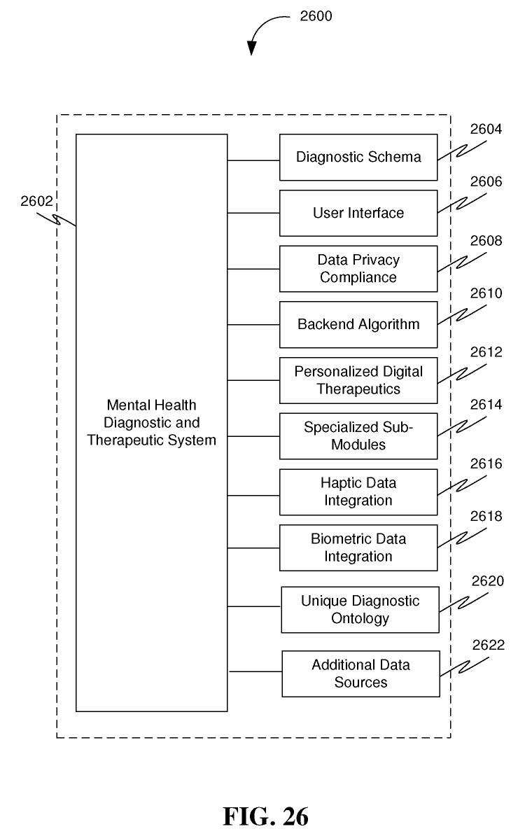

Further, referring to FIG. 26, in some embodiments, the future extension provisions may comprise future extension provisions which may include future extensions mental health diagnostic and therapeutic system 2602 into diagnostic schema 2604, user interface 2606, data privacy compliance 2608, backend algorithm 2610, personalized digital therapeutics 2612, specialized sub modules 2614, haptic data integration 2616, biometric data integration 2618, unique diagnostic ontology 2620 and/or additional data sources 2622.

1. User Interface & Interactive Data Collection System: The User Interface serves as the front end of the system where users interact to provide their medical history and other relevant information. The Interactive Data Collection System facilitates this process by presenting a blend of standardized and proprietary questions, and allowing various data input methods like text, voice, video, and image.

2. Proprietary Diagnostic Schema: The collected data is then fed into the Proprietary Diagnostic Schema, which organizes and categorizes the information in a structured format to facilitate further analysis.

3. Machine Learning Module: The Machine Learning Module interacts with the Proprietary Diagnostic Schema to dynamically refine the diagnostic criteria based on accumulated data, user feedback, and external medical databases. This interaction ensures the diagnostic schema evolves over time to improve diagnostic accuracy and treatment recommendations.

4. Specialized Sub-Modules: Specialized Sub-Modules are designed for different medical specialties and interact with the Proprietary Diagnostic Schema to process and analyze the data relevant to their respective specialties. This cross-specialty interaction enhances the holistic understanding of the user’s health conditions.

5. Backend Algorithm: The Backend Algorithm processes the structured data from the Proprietary Diagnostic Schema to generate a pre-diagnosis and/or diagnosis for clinician review. It also proposes personalized digital therapeutics based on the diagnostic outcome.

6. Personalized Digital Therapeutics Module: This module works in tandem with the Backend Algorithm to propose tailored digital therapeutic interventions based on the pre-diagnosis and/or diagnosis and the evolving diagnostic schema.

7. Data Privacy Compliance System: Throughout the process, the Data Privacy Compliance System ensures that all data collected, stored, and processed is handled securely in compliance with healthcare data privacy regulations.

8. External Medical Databases Integration: The system can interact with External Medical Databases to incorporate additional medical knowledge and data which can be utilized by the Machine Learning Module and the Backend Algorithm to refine the diagnostic schema and improve diagnostic and therapeutic outcomes.

9. Future Extension Provisions: Provisions are made within the system to allow for future extensions, such as the integration of unique diagnostic ontology and additional data sources, ensuring the system remains adaptive to evolving medical knowledge.

10. Continuous Improvement Mechanism: This mechanism works across all components to gather feedback, monitor performance, and make necessary refinements to enhance diagnostic accuracy, treatment efficacy, and overall user experience over time.

Proprietary diagnostic schema organizes and categorizes the collected medical history data in a structured format that facilitates further analysis. It serves as the foundation for the Backend Algorithm and Machine Learning Module to operate effectively, ensuring accurate preliminary diagnoses and personalized digital therapeutic proposals.

Machine learning module autonomously analyzes accumulated data, user feedback, and external medical databases to refine the diagnostic criteria dynamically. It continually refines the Proprietary Diagnostic Schema, enhancing the diagnostic accuracy and therapeutic effectiveness of the system over time. User interface & interactive data collection system provide an intuitive platform for users to input their medical history data using various methods like text, voice, video, and image. They funnel essential data into the Proprietary Diagnostic Schema, facilitating the interactive collection and structured storage of medical history information.

Backend Algorithm processes the structured data to generate a pre-diagnosis and/or diagnosis for clinician review and proposes personalized digital therapeutics. It acts as a bridge between the diagnostic schema and the personalized digital therapeutics module, translating structured data into actionable diagnostic and therapeutic proposals.

Specialized sub-modules cater to a specific medical specialty, analyzing the data relevant to its domain. They enrich the Proprietary Diagnostic Schema with specialized insights, enabling a more holistic understanding and treatment of mental health conditions.

Data privacy compliance system ensures all data handling practices comply with healthcare data privacy regulations. It provides a secure environment for all other components to operate, ensuring user trust and legal compliance.

Future extension provisions allows for the system's adaptation to evolving medical knowledge by incorporating new diagnostic ontology and data sources. It ensures the long-term relevance and effectiveness of the entire system by allowing for continuous adaptation and improvement.

Personalized digital therapeutics module creates tailored therapeutic interventions based on the pre-diagnosis and/or diagnosis and the evolving diagnostic schema. It works hand-in hand with the Backend Algorithm to provide a seamless transition from diagnosis to treatment, enhancing the overall user experience and treatment efficacy.

External medical databases integration facilitates the integration of additional medical knowledge and data into the system. It enriches the machine learning module and diagnostic schema with external insights, contributing to the continuous improvement of diagnostic accuracy and therapeutic effectiveness.

Continuous improvement mechanism gathers feedback and monitors the system's performance to make necessary refinements. It operates across all components, ensuring a continuous cycle of improvement, enhancing diagnostic accuracy, treatment efficacy, and overall user experience over time.

The harmonious interaction among these components, each performing its unique function while collaboratively working towards the common goal, embodies the essence of the invention. This synergistic operation enables the system to provide a more personalized, dynamic, and integrated approach to mental health diagnostics and digital therapeutics, significantly improving the quality of mental health care delivery in the digital realm.

Creating this invention entails a structured, step-by-step approach that combines technical development, iterative testing, and continuous improvement. Here's a detailed outline of how to make your invention:

### 1. Initial Research and Planning:

Conduct a comprehensive analysis of existing digital diagnostic and therapeutic tools within the mental health domain and identify their limitations.

Gather a multidisciplinary team of experts including software developers, machine learning engineers, medical professionals, and user experience designers.

### 2. Development of Proprietary Diagnostic Schema:

Design the proprietary diagnostic schema that will serve as the backbone for data collection and analysis.

Collaborate with medical professionals to ensure the schema is medically sound and comprehensive.

### 3. Machine Learning Module Development:

Design and develop the machine learning algorithms that will dynamically refine the diagnostic schema.

Ensure the algorithms are capable of processing large volumes of data and can learn and adapt over time.

### 4. User Interface Design and Development:

Design a user-friendly interface for data collection, ensuring ease of use across diverse patient demographics.

Develop the interactive data collection system within the user interface, incorporating various data input methods.

### 5. Backend Algorithm Development:

Design and develop the backend algorithm to process the collected data, generate a pre-diagnosis and/or diagnosis, and propose personalized digital therapeutics.

Ensure the algorithm is robust, efficient, and can interact seamlessly with other system components.

### 6. Specialized Sub-Modules Development:

Develop specialized sub-modules for different medical specialties, ensuring they are tailored to process relevant data effectively.

Collaborate with medical experts to validate the sub-modules’ effectiveness in their respective domains.

### 7. Data Privacy Compliance Implementation:

Implement robust data security measures to ensure compliance with healthcare data privacy regulations.

Consult with legal experts to validate the data privacy compliance of the system.

### 8. Integration of External Medical Databases:

Establish connections with external medical databases to enrich the machine learning module and diagnostic schema with additional medical knowledge and data.

### 9. Testing and Refinement:

Conduct rigorous testing of the system in a controlled environment to identify bugs, errors, and areas of improvement.

Gather feedback from a select group of users and medical professionals to refine the system components.

### 10. Deployment of Future Extension Provisions:

Implement provisions within the system for future extensions to incorporate new diagnostic ontology and additional data sources.

### 11. Initial Deployment:

Deploy the system in a controlled or limited environment to gather real world feedback and monitor system performance.

Make necessary refinements based on feedback and performance metrics.

### 12. Continuous Improvement:

Post-deployment, establish a continuous improvement mechanism to gather user feedback, monitor system performance, and make necessary refinements.

Engage with medical professionals to ensure the system stays updated with emerging medical knowledge.

### 13. Broader Deployment:

Once the system has been refined and proven effective in a controlled environment, plan for a broader deployment to make the system available to a wider user base.

### 14. Monitoring and Long-Term Improvement:

Continuously monitor the system's performance, gather user feedback, and collaborate with medical professionals to ensure long-term effectiveness and relevance.

This structured approach ensures a systematic development, testing, refinement, and deployment of the invention, aligning it with the overarching goal of providing personalized, accurate, and effective digital mental health diagnostics and therapeutics.

The system provides a novel and comprehensive system and method of the interactive collection and analysis of medical history across diverse medical specialties, with a significant emphasis on mental health. By employing a proprietary diagnostic schema dynamically refined through machine learning techniques, the system facilitates more personalized diagnoses and the provision of tailored digital therapeutics. This innovation aims to significantly improve diagnostic accuracy, treatment efficacy, and the overall patient experience in the mental health domain, thereby addressing the limitations of existing digital diagnostic and therapeutic tools. The system's holistic approach, ease of use, and robust data privacy compliance further underscore its potential to revolutionize mental health care in the digital age.

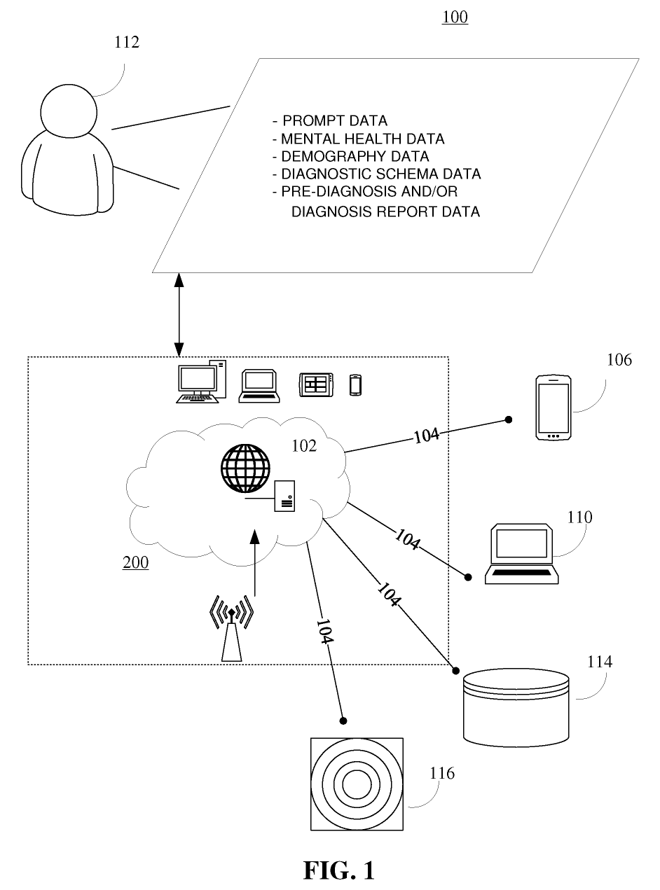

FIG. 1 is an illustration of an online platform 100 consistent with various embodiments of the present disclosure. By way of non-limiting example, the online platform 100 may be hosted on a centralized server 102, such as, for example, a cloud computing service. The centralized server 102 may communicate with other network entities, such as, for example, a mobile device 106 (such as a smartphone, a laptop, a tablet computer etc.), other electronic devices 110 (such as desktop computers, server computers etc.), databases 114, and sensors 116 over a communication network 104, such as, but not limited to, the Internet. Further, users of the online platform 100 may include relevant parties such as, but not limited to, end-users, administrators, service providers, service consumers and so on. Accordingly, in some instances, electronic devices operated by the one or more relevant parties may be in communication with the platform.

A user 112, such as the one or more relevant parties, may access online platform 100 through a web based software application or browser. The web based software application may be embodied as, for example, but not be limited to, a website, a web application, a desktop application, and a mobile application compatible with a computing device 200.

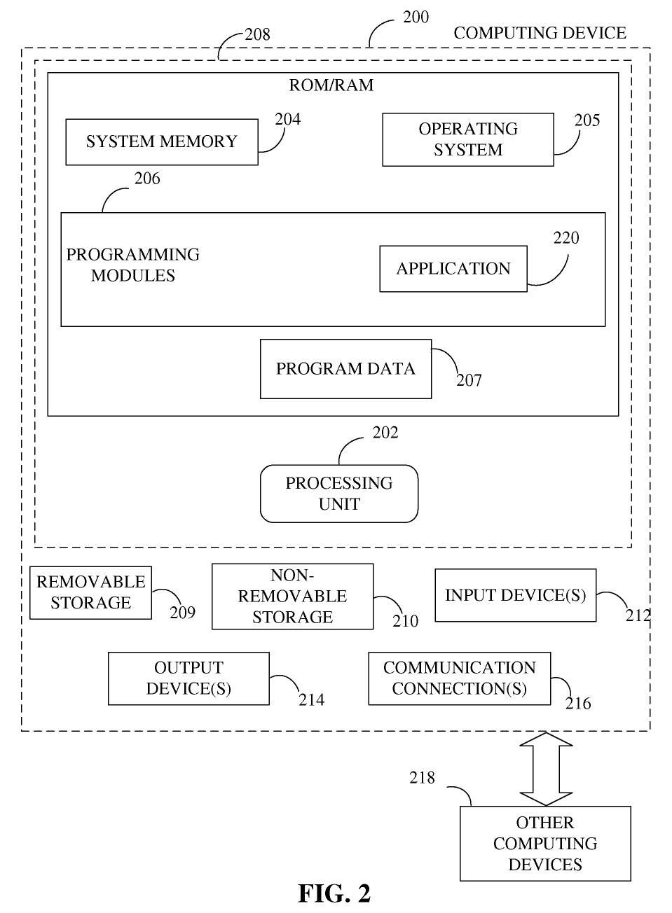

With reference to FIG. 2, a system consistent with an embodiment of the disclosure may include a computing device or cloud service, such as computing device 200. In a basic configuration, computing device 200 may include at least one processing unit 202 and a system memory 204. Depending on the configuration and type of computing device, system memory 204 may comprise, but is not limited to, volatile (e.g. random-access memory (RAM)), non-volatile (e.g. read-only memory (ROM)), flash memory, or any combination. System memory 204 may include operating system 205, one or more programming modules 206, and may include a program data 207. Operating system 205, for example, may be suitable for controlling computing device 200’s operation. In one embodiment, programming modules 206 may include image-processing module, machine learning module. Furthermore, embodiments of the disclosure may be practiced in conjunction with a graphics library, other operating systems, or any other application program and is not limited to any particular application or system. This basic configuration is illustrated in FIG. 2 by those components within a dashed line 208.

Computing device 200 may have additional features or functionality. For example, computing device 200 may also include additional data storage devices (removable and/or non-removable) such as, for example, magnetic disks, optical disks, or tape. Such additional storage is illustrated in FIG. 2 by a removable storage 209 and a non-removable storage 210. Computer storage media may include volatile and non-volatile, removable and non-removable media implemented in any method or technology for storage of information, such as computer-readable instructions, data structures, program modules, or other data. System memory 204, removable storage 209, and non-removable storage 210 are all computer storage media examples (i.e., memory storage.) Computer storage media may include, but is not limited to, RAM, ROM, electrically erasable read-only memory (EEPROM), flash memory or other memory technology, CD-ROM, digital versatile disks (DVD) or other optical storage, magnetic cassettes, magnetic tape, magnetic disk storage or other magnetic storage devices, or any other medium which can be used to store information and which can be accessed by computing device 200. Any such computer storage media may be part of device 200. Computing device 200 may also have input device(s) 212 such as a keyboard, a mouse, a pen, a sound input device, a touch input device, a location sensor, a camera, a biometric sensor, etc. Output device(s) 214 such as a display, speakers, a printer, etc. may also be included. The aforementioned devices are examples and others may be used.

Computing device 200 may also contain a communication connection 216 that may allow device 200 to communicate with other computing devices 218, such as over a network in a distributed computing environment, for example, an intranet or the Internet. Communication connection 216 is one example of communication media. Communication media may typically be embodied by computer readable instructions, data structures, program modules, or other data in a modulated data signal, such as a carrier wave or other transport mechanism, and includes any information delivery media. The term “modulated data signal” may describe a signal that has one or more characteristics set or changed in such a manner as to encode information in the signal. By way of example, and not limitation, communication media may include wired media such as a wired network or direct-wired connection, and wireless media such as acoustic, radio frequency (RF), infrared, and other wireless media. The term computer readable media as used herein may include both storage media and communication media.

As stated above, a number of program modules and data files may be stored in system memory 204, including operating system 205. While executing on processing unit 202, programming modules 206 (e.g., application 220 such as a media player) may perform processes including, for example, one or more stages of methods, algorithms, systems, applications, servers, databases as described above. The aforementioned process is an example, and processing unit 202 may perform other processes. Other programming modules that may be used in accordance with embodiments of the present disclosure may include machine learning applications.

Generally, consistent with embodiments of the disclosure, program modules may include routines, programs, components, data structures, and other types of structures that may perform particular tasks or that may implement particular abstract data types. Moreover, embodiments of the disclosure may be practiced with other computer system configurations, including hand-held devices, general purpose graphics processor-based systems, multiprocessor systems, microprocessor-based or programmable consumer electronics, application specific integrated circuit-based electronics, minicomputers, mainframe computers, and the like. Embodiments of the disclosure may also be practiced in distributed computing environments where tasks are performed by remote processing devices that are linked through a communications network. In a distributed computing environment, program modules may be located in both local and remote memory storage devices.

Furthermore, embodiments of the disclosure may be practiced in an electrical circuit comprising discrete electronic elements, packaged or integrated electronic chips containing logic gates, a circuit utilizing a microprocessor, or on a single chip containing electronic elements or microprocessors. Embodiments of the disclosure may also be practiced using other technologies capable of performing logical operations such as, for example, AND, OR, and NOT, including but not limited to mechanical, optical, fluidic, and quantum technologies. In addition, embodiments of the disclosure may be practiced within a general-purpose computer or in any other circuits or systems.

Embodiments of the disclosure, for example, may be implemented as a computer process (method), a computing system, or as an article of manufacture, such as a computer program product or computer readable media. The computer program product may be a computer storage media readable by a computer system and encoding a computer program of instructions for executing a computer process. The computer program product may also be a propagated signal on a carrier readable by a computing system and encoding a computer program of instructions for executing a computer process. Accordingly, the present disclosure may be embodied in hardware and/or in software (including firmware, resident software, micro-code, etc.). In other words, embodiments of the present disclosure may take the form of a computer program product on a computer-usable or computer-readable storage medium having computer-usable or computer-readable program code embodied in the medium for use by or in connection with an instruction execution system. A computer-usable or computer-readable medium may be any medium that can contain, store, communicate, propagate, or transport the program for use by or in connection with the instruction execution system, apparatus, or device.

The computer-usable or computer-readable medium may be, for example but not limited to, an electronic, magnetic, optical, electromagnetic, infrared, or semiconductor system, apparatus, device, or propagation medium. More specific computer-readable medium examples (a non-exhaustive list), the computer-readable medium may include the following: an electrical connection having one or more wires, a portable computer diskette, a random-access memory (RAM), a read-only memory (ROM), an erasable programmable read-only memory (EPROM or Flash memory), an optical fiber, and a portable compact disc read-only memory (CD-ROM). Note that the computer-usable or computer-readable medium could even be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory.

Embodiments of the present disclosure, for example, are described above with reference to block diagrams and/or operational illustrations of methods, systems, and computer program products according to embodiments of the disclosure. The functions/acts noted in the blocks may occur out of the order as shown in any flowchart. For example, two blocks shown in succession may in fact be executed substantially concurrently or the blocks may sometimes be executed in the reverse order, depending upon the functionality/acts involved.

While certain embodiments of the disclosure have been described, other embodiments may exist. Furthermore, although embodiments of the present disclosure have been described as being associated with data stored in memory and other storage mediums, data can also be stored on or read from other types of computer-readable media, such as secondary storage devices, like hard disks, solid state storage (e.g., USB drive), or a CD-ROM, a carrier wave from the Internet, or other forms of RAM or ROM. Further, the disclosed methods’ stages may be modified in any manner, including by reordering stages and/or inserting or deleting stages, without departing from the disclosure.

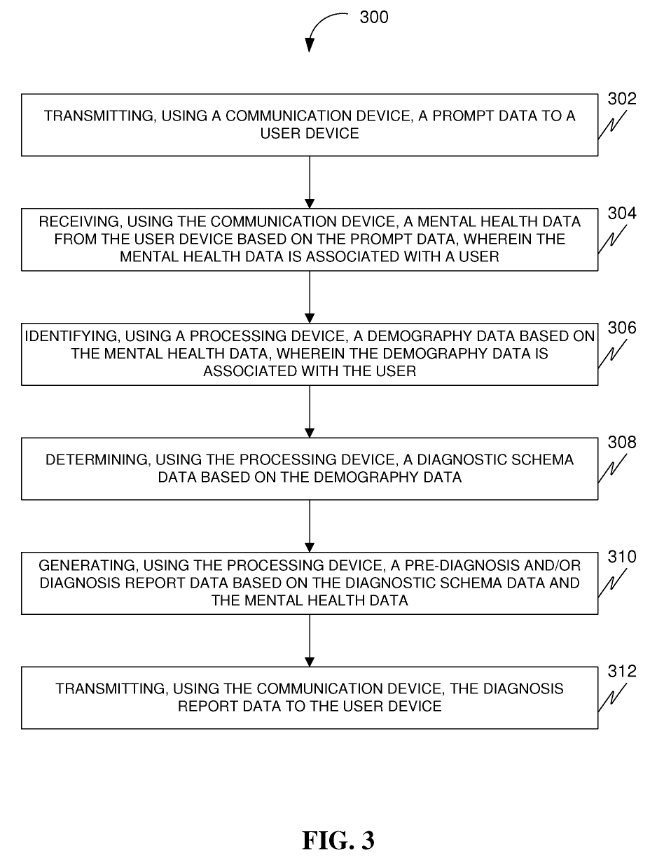

FIG. 3 illustrates a flowchart of a method 300 of facilitating AI-driven refinement of mental health diagnostics and personalized treatment, in accordance with some embodiments.

Accordingly, the method 300 may include a step 302 of transmitting, using a communication device 702, a prompt data to a user device. Further, the method 300 may include a step 304 of receiving, using the communication device 702, a mental health data from the user device based on the prompt data. Further, the mental health data may be associated with a user. Further, the method 300 may include a step 306 of identifying, using a processing device 704, a demography data based on the mental health data. Further, the demography data may be associated with the user. Further, the method 300 may include a step 308 of determining, using the processing device 704, a diagnostic schema data based on the demography data. Further, the method 300 may include a step 310 of generating, using the processing device 704, a pre-diagnosis and/or diagnosis report data based on the diagnostic schema data and the mental health data. Further, the method 300 may include a step 312 of transmitting, using the communication device 702, the pre-diagnosis and/or diagnosis report data to the user device.

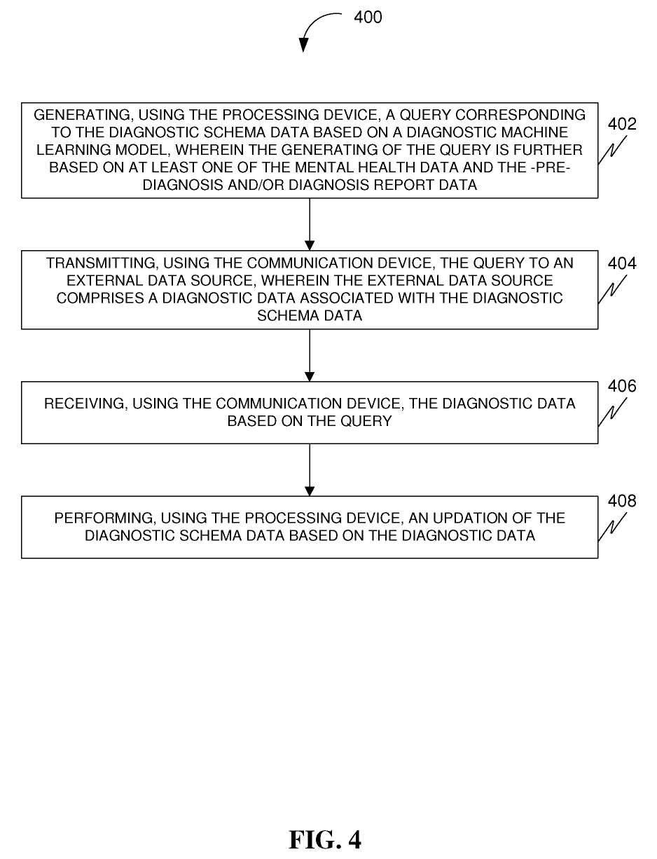

FIG. 4 illustrates a flowchart of a method 400 of facilitating AI-driven refinement of mental health diagnostics and personalized treatment including performing, using the processing device 704, an updation of the diagnostic schema data, in accordance with some embodiments.

Further, in some embodiments, the method 400 further may include a step 402 of generating, using the processing device 704, a query corresponding to the diagnostic schema data based on a diagnostic machine learning model. Further, the generating of the query may be further based on one or more of the mental health data and the pre-diagnosis and/or diagnosis report data. Further, in some embodiments, the method 400 further may include a step 404 of transmitting, using the communication device 702, the query to an external data source. Further, the external data source includes a diagnostic data associated with the diagnostic schema data. Further, in some embodiments, the method 400 further may include a step 406 of receiving, using the communication device 702, the diagnostic data based on the query. Further, in some embodiments, the method 400 further may include a step 408 of performing, using the processing device 704, an updation of the diagnostic schema data based on the diagnostic data.

In some embodiments, the method 300 may further include encrypting a part of the pre-diagnosis and/or diagnosis report data to obtain an encrypted data. Further, the pre-diagnosis and/or diagnosis report data includes the encrypted data in place of the part of the pre-diagnosis and/or diagnosis report data.

In some embodiments, the prompt data represents an activity performable by the user. Further, the mental health data may be generated based on a performance of the activity.

In some embodiments, the pre-diagnosis and/or diagnosis report data includes a pre-diagnosis and/or diagnosis data. Further, the pre-diagnosis and/or diagnosis data may be transmitted to a clinician device associated with a clinician.

In some embodiments, the method 300 may further include determining, using the processing device 704, two or more medical domain indicators corresponding to the mental health data based on a domain machine learning model. Further, the determining includes performing a first analysis of the mental health data.

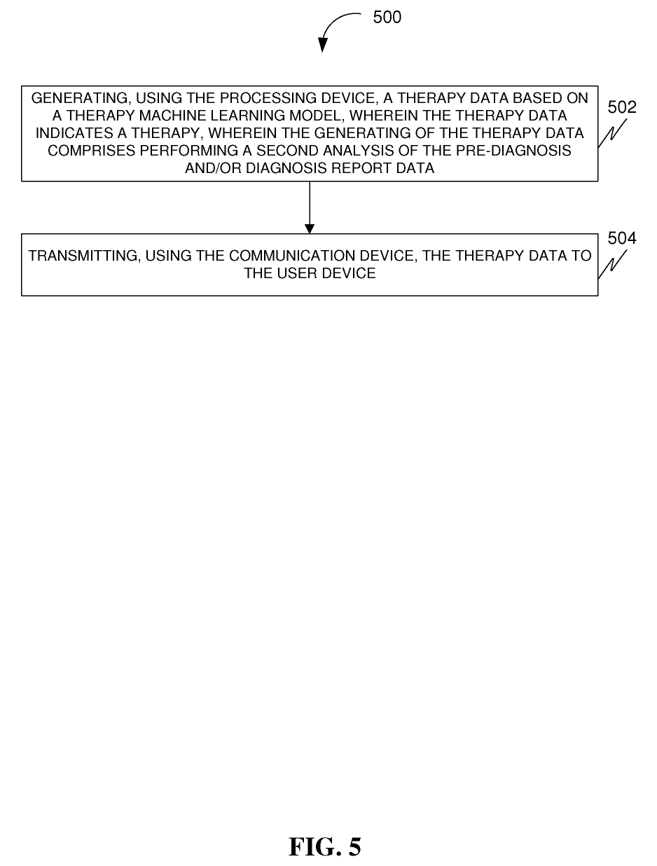

FIG. 5 illustrates a flowchart of a method 500 of facilitating AI-driven refinement of mental health diagnostics and personalized treatment including generating, using the processing device 704, a therapy data, in accordance with some embodiments.

Further, in some embodiments, the method 500, further may include a step 502 of generating, using the processing device 704, a therapy data based on a therapy machine learning model. Further, in some embodiments, the machine learning module may include a multi-modal deep learning module. Further, at least one of the pre-diagnosis and/or diagnosis report data may correspond to a plurality of modalities, such as for example, text data, audio data, image data, video data, medical instrument data and sensor data. Further, the therapy data indicates a therapy. Further, the generating of the therapy data includes performing a second analysis of the pre-diagnosis and/or diagnosis report data. Further, in some embodiments, the method 500, further may include a step 504 of transmitting, using the communication device 702, the therapy data to the user device.

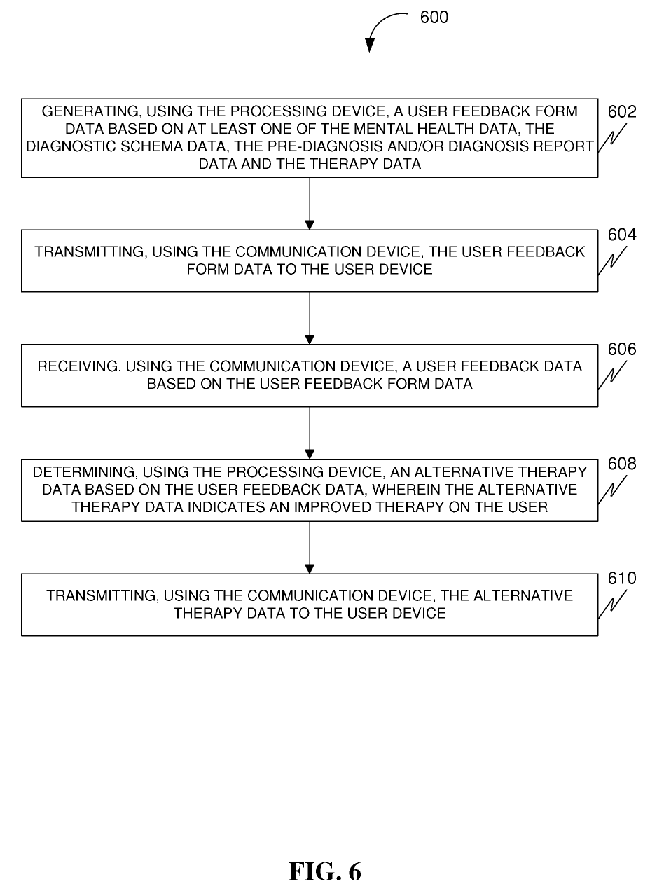

FIG. 6 illustrates a flowchart of a method 600 of facilitating AI-driven refinement of mental health diagnostics and personalized treatment including determining, using the processing device 704, an alternative therapy data, in accordance with some embodiments.

Further, in some embodiments, the method 600, further may include a step 602 of generating, using the processing device 704, a user feedback form data based on one or more of the mental health data, the diagnostic schema data, the pre-diagnosis and/or diagnosis report data and the therapy data. Further, in some embodiments, the method 600, further may include a step 604 of transmitting, using the communication device 702, the user feedback form data to the user device. Further, in some embodiments, the method 600, further may include a step 606 of receiving, using the communication device 702, a user feedback data based on the user feedback form data. Further, in some embodiments, the method 600, further may include a step 608 of determining, using the processing device 704, an alternative therapy data based on the user feedback data. Further, the alternative therapy data indicates an improved therapy on the user. Further, in some embodiments, the method 600, further may include a step 610 of transmitting, using the communication device 702, the alternative therapy data to the user device.

In some embodiments, the mental health data includes a lifestyle data representing a manner of living of the user.

In some embodiments, the method 300 may further include generating, using the processing device 704, the prompt data. Further, the mental health data includes a first mental health data and a second mental health data. Further, the prompt data includes a first prompt data and a second prompt data. Further, the first mental health data may be received in response to the first prompt data and the second mental health data may be received in response to the second prompt data. Further, the generating of the second prompt data may be based on the first mental health data.

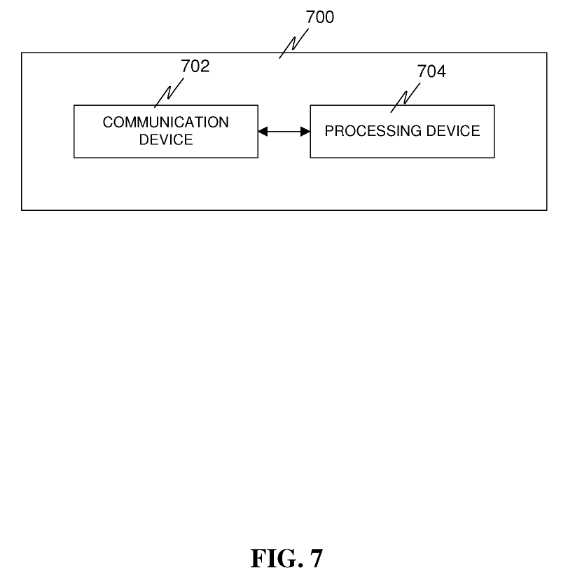

FIG. 7 illustrates a block diagram of a system 700 of facilitating AI-driven refinement of mental health diagnostics and personalized treatment, in accordance with some embodiments.

Accordingly, the system 700 may include a communication device 702. Further, the communication device 702 may be configured for transmitting a prompt data to a user device. Further, the communication device 702 may be configured for receiving a mental health data from the user device based on the prompt data. Further, the mental health data may be associated with a user. Further, the communication device 702 may be configured for transmitting a pre-diagnosis and/or diagnosis report data to the user device. Further, the system 700 may include a processing device 704. Further, the processing device 704 may be configured for identifying a demography data associated with the user based on the mental health data. Further, the processing device 704 may be configured for identifying a diagnostic schema data based on the demography data. Further, the processing device 704 may be configured for generating the pre-diagnosis and/or diagnosis report data based on the diagnostic schema data.

Further, in some embodiments, the processing device 704 may be further configured for generating a query corresponding to the diagnostic schema data based on a diagnostic machine learning model. Further, the generating of the query may be further based on one or more of the mental health data and the pre-diagnosis and/or diagnosis report data. Further, the processing device 704 may be further configured for performing an updation of the diagnostic schema data based on the diagnostic data. Further, the communication device 702 may be further configured for transmitting the query to an external data source. Further, the external source includes the diagnostic data related to the diagnostic schema data. Further, the communication device 702 may be further configured for receiving the diagnostic data from the external data source based on the query.

In some embodiments, the processing device 704 may be further configured for encrypting a part of the pre-diagnosis and/or diagnosis report data to obtain an encrypted data. Further, the pre-diagnosis and/or diagnosis report data includes the encrypted data in place of the part of the pre-diagnosis and/or diagnosis report data.

In some embodiments, the prompt data represents an activity. Further, the activity may be performable by the user. Further, the mental health data may be generated based on a performance of the activity.

In some embodiments, the pre-diagnosis and/or diagnosis report data includes a pre-diagnosis and/or diagnosis data. Further, the communication device 702 may be further configured for transmitting the pre-diagnosis and/or diagnosis data to a clinician device associated with a clinician.

In some embodiments, the processing device 704 may be further configured for determining two or more medical domain indicators corresponding to the mental health data based on a domain machine learning model. Further, the determining includes performing a first analysis of the mental health data.

In some embodiments, the processing device 704 may be further configured for generating a therapy data based on a therapy machine learning model. Further, the therapy data indicates a therapy. Further, the generating includes performing a second analysis of the pre-diagnosis and/or diagnosis report data. Further, the communication device 702 may be further configured for transmitting the therapy data to the user device.

Further, in some embodiments, the processing device 704 may be further configured for generating a user feedback form data based on one or more of the mental health data, the diagnostic schema data, the pre-diagnosis and/or diagnosis report data and the therapy data. Further, the processing device 704 may be further configured for determining an alternative therapy data based on a user feedback data. Further, the alternative therapy data indicates an improved therapy on the user. Further, the communication device 702 may be further configured for transmitting the user feedback form data to the user device. Further, the communication device 702 may be further configured for receiving a user feedback data based on the user feedback form data. Further, the communication device 702 may be further configured for transmitting the alternative therapy data to the user device.

In some embodiments, the mental health data includes a lifestyle data representing a manner of living of the user.

In some embodiments, the processing device 704 may be further configured for generating the prompt data. Further, the mental health data includes a first mental health data and a second mental health data. Further, the prompt data includes a first prompt data and a second prompt data. Further, the first mental health data may be received in response to the first prompt data and the second mental health data may be received in response to the second prompt data. Further, the generating of the second prompt data may be based on the first mental health data.

In some embodiments, the method 300 may further include updating, using the processing device 704, the domain machine learning model based on one or more of the mental health data and the pre-diagnosis and/or diagnosis report data.

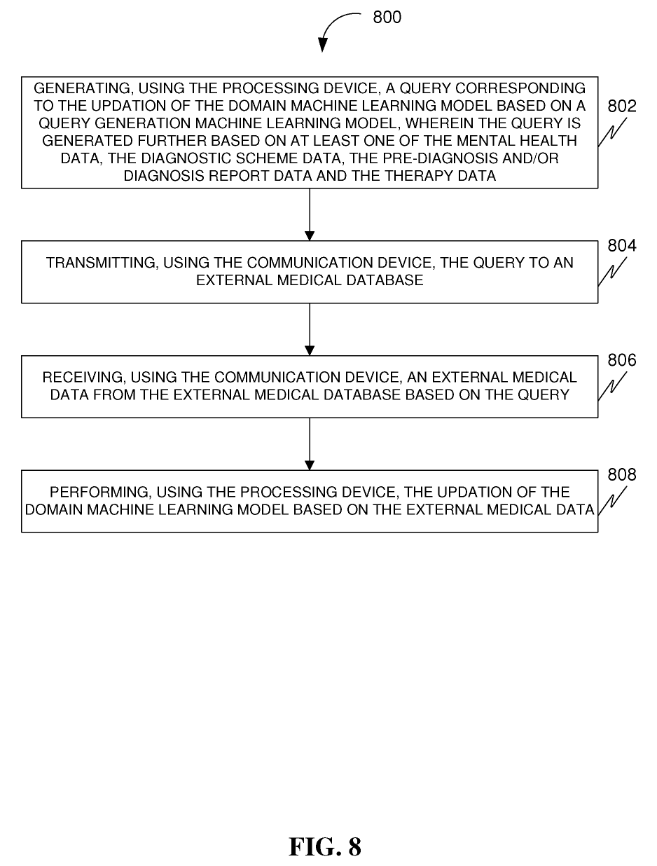

FIG. 8 illustrates a flowchart of a method 800 of facilitating AI-driven refinement of mental health diagnostics and personalized treatment including performing, using the processing device 704, the updation of the domain machine learning model, in accordance with some embodiments.

Further, in some embodiments, the method 800 further may include a step 802 of generating, using the processing device 704, a query corresponding to the updation of the domain machine learning model based on a query generation machine learning model. Further, the query may be generated further based on one or more of the mental health data, the diagnostic scheme data, the pre-diagnosis and/or diagnosis report data and the therapy data. Further, in some embodiments, the method 800 further may include a step 804 of transmitting, using the communication device 702, the query to an external medical database. Further, in some embodiments, the method 800 further may include a step 806 of receiving, using the communication device 702, an external medical data from the external medical database based on the query. Further, in some embodiments, the method 800 further may include a step 808 of performing, using the processing device 704, the updation of the domain machine learning model based on the external medical data.

In some embodiments, the activity includes an interpretation of an image, may. Further, the prompt data includes an image data representing the image, may. Further, the user device includes a presentation device which may be configured to present the image data. Further, the user device further includes one or more of an input device and a user communication device. Further, the input device may be configured to receive a response provided by the user based on the presentation of the image data. Further, the user communication device may be configured to receive the response from an external device which may be configured to generate the response provided by the user. Further, the user device further includes a user processing device which may be configured to generate an interpretation data based on the response of the user. Further, the mental health data includes the interpretation data.

In some embodiments, the activity includes an emotion expression task. Further, the prompt data includes a scenario data representing a scenario. Further, the user device includes a presentation device which may be configured to present the scenario data. Further, an emotion may be acted out by the user based on the presentation of the scenario data. Further, the user device further includes an emotion sensor which may be configured to detect an emotion data associated with the emotion. Further, the mental health data includes the emotion data.

In some embodiments, the activity includes a journaling task. Further, the user device includes a presentation device which may be configured to present a journal entry data associated with the journaling task. Further, the user device includes an input device which may be configured to receive a text data from the user based on the presentation of the journal entry data. Further, the user device further includes a user processing device which may be configured to generate a journal data. Further, the generation of the journal data may be further based on an analysis of the text data. Further, the mental health data includes the journal data.

In some embodiments, the activity includes a speech task. Further, the user device includes a presentation device which may be configured to present a speech entry data associated with the speech task. Further, the user device includes an input device which may be configured to receive speech data from the user based on the presentation of the speech entry data. Further, the user device includes a voice sensor which may be configured to detect one or more of a voice pitch, a tone and a speech pattern of the speech data. Further, the user device further includes a user processing device which may be configured to generate an evaluated speech data. Further, the generation of the evaluated speech data may be further based on an analysis of the speech data. Further, the mental health data includes the evaluated speech data.

In some embodiments, the activity includes a focusing task. Further, the prompt data includes a scenario data representing a scenario. Further, the user device includes a presentation device which may be configured to present the scenario data. Further, the user device further includes an eye-tracking sensor which may be configured to detect a focus ability of the user. Further, the user device further includes a user processing device which may be configured to generate a focus data based on the focusing ability. Further, the mental health data includes the focus data.

In some embodiments, the activity includes a meditation exercise. Further, the prompt data includes a meditation exercise data representing a collection of meditation exercises. Further, the user device includes a presentation device which may be configured to present the collection of meditation exercises. Further, a meditation exercise may be performable by the user based on the presentation of the collection of meditation exercises. Further, the user device further includes an optical sensor which may be configured to detect a facial expression corresponding to an expression data associated with the meditation exercise. Further, the mental health data includes the expression data.

In some embodiments, the activity includes a meditation exercise. Further, the prompt data includes a meditation exercise data representing a collection of meditation exercises. Further, the user device includes a presentation device which may be configured to present the collection of meditation exercises. Further, a meditation exercise may be performable by the user based on the presentation of the collection of meditation exercises. Further, the user device further includes a thermal sensor which may be configured to detect a breathing pattern corresponding to a breathing data associated with the meditation exercise. Further, the mental health data includes the breathing data.

In some embodiments, the activity includes a physical exercise. Further, the prompt data includes a physical exercise data representing a collection of physical exercises. Further, the user device includes a presentation device which may be configured to present the collection of physical exercises. Further, a physical exercise may be performed by the user based on the presentation of the collection of physical exercises. Further, the user device further includes a motion sensor which may be configured to detect an exercise data associated with the physical exercise. Further, the mental health data includes the exercise data.

In some embodiments, the prompt data represents a cognitive assessment. Further, the cognitive assessment may be undertaken by a user associated with the user device. Further, the mental health data may be generated based on the performance of the cognitive assessment.

In some embodiments, the user device may include at least one sensor configured to generate the mental health data based on the activity. Further, in an instance, the at least one sensor may include each of a camera and a microphone.

In some embodiments, the therapy data includes indication of one or more of a psychological therapy and a physical therapy.

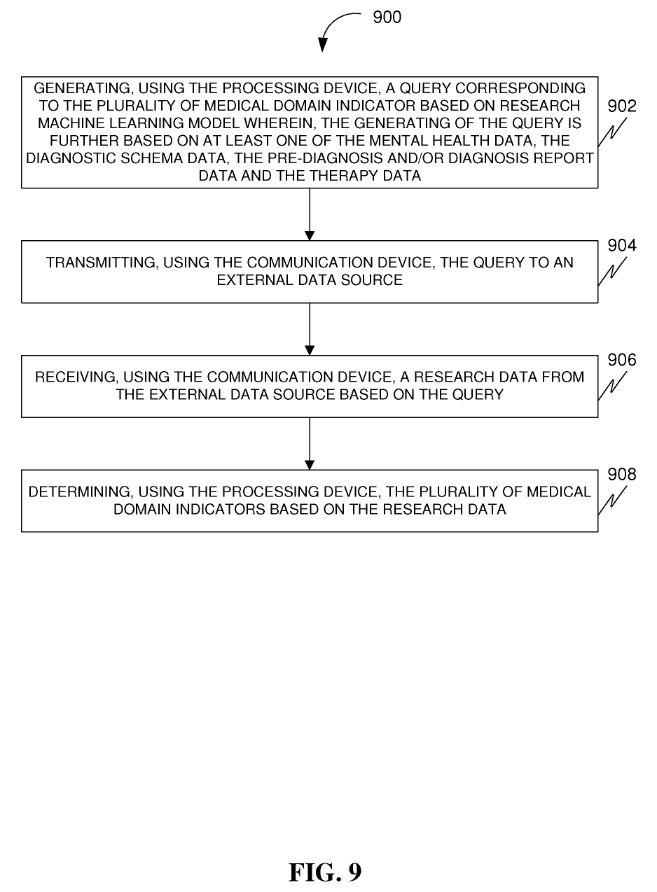

FIG. 9 illustrates a flowchart of a method 900 of facilitating AI-driven refinement of mental health diagnostics and personalized treatment including determining, using the processing device 704, the plurality of medical domain indicators, in accordance with some embodiments.

Further, in some embodiments, the method 900 further may include a step 902 of generating, using the processing device 704, a query corresponding to the two or more medical domain indicators based on research machine learning model. Further, the generating of the query may be further based on one or more of the mental health data, the diagnostic schema data, the pre-diagnosis and/or diagnosis report data and the therapy data. Further, in some embodiments, the method 900 further may include a step 904 of transmitting, using the communication device 702, the query to an external data source. Further, in some embodiments, the method 900 further may include a step 906 of receiving, using the communication device 702, a research data from the external data source based on the query. Further, in some embodiments, the method 900 further may include a step 908 of determining, using the processing device 704, the two or more medical domain indicators based on the research data.

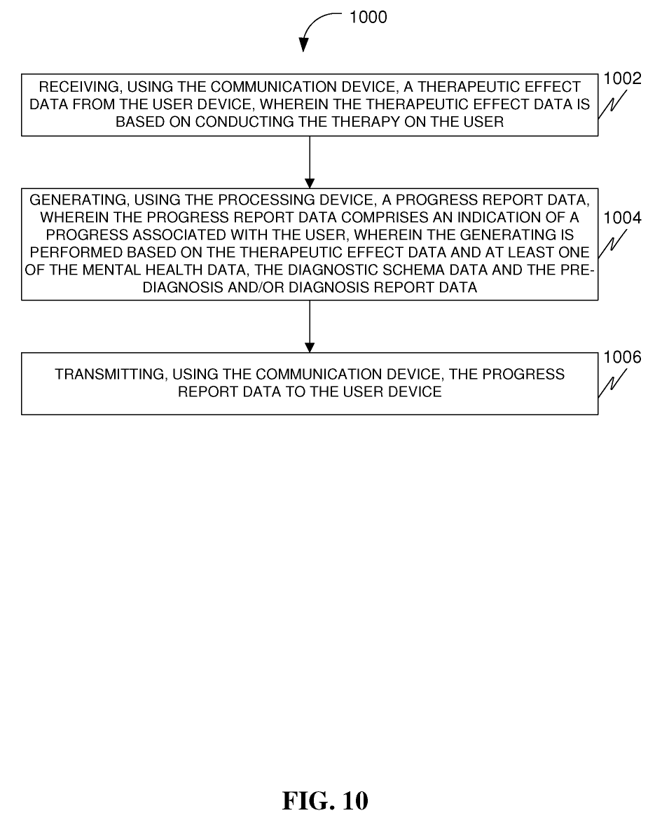

FIG. 10 illustrates a flowchart of a method 1000 of facilitating AI-driven refinement of mental health diagnostics and personalized treatment including generating, using the processing device 704, a progress report data, in accordance with some embodiments.

Further, in some embodiments, the method 1000 further may include a step 1002 of receiving, using the communication device 702, a therapeutic effect data from the user device. Further, the therapeutic effect data may be based on conducting the therapy on the user. Further, in some embodiments, the method 1000 further may include a step 1004 of generating, using the processing device 704, a progress report data. Further, the progress report data includes an indication of a progress associated with the user. Further, the generating may be performed based on the therapeutic effect data and one or more of the mental health data, the diagnostic schema data and the pre-diagnosis and/or diagnosis report data. Further, in some embodiments, the method 1000 further may include a step 1006 of transmitting, using the communication device 702, the progress report data to the user device.

In some embodiments, the pre-diagnosis and/or diagnosis report data includes a contentment index data based on the mental health data. Further, the contentment index data represents a mental health of the user associated with the mental health data.

In some embodiments, the encrypting may be in compliance with one or more healthcare regulations.

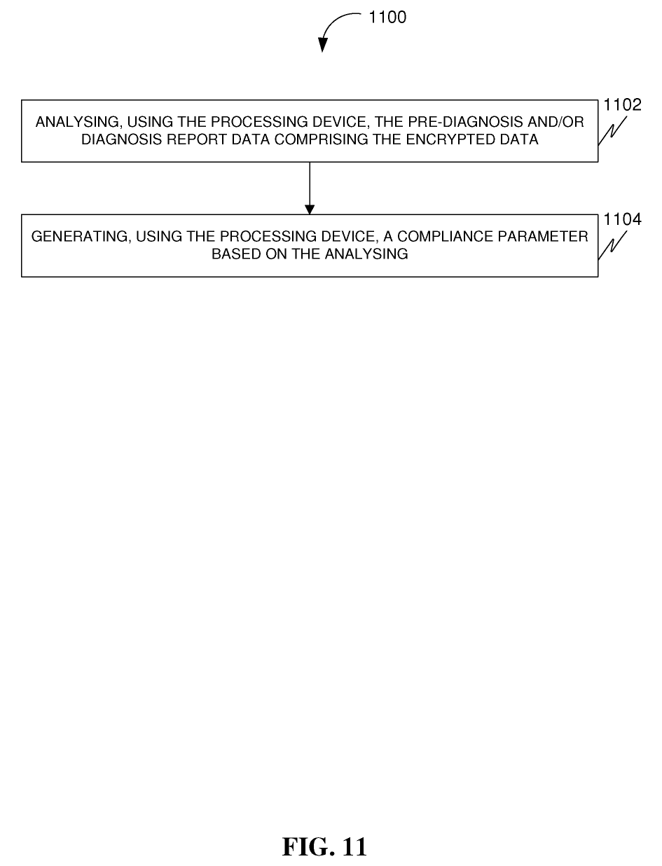

FIG. 11 illustrates a flowchart of a method 1100 of facilitating AI-driven refinement of mental health diagnostics and personalized treatment including generating, using the processing device 704, a compliance parameter, in accordance with some embodiments.

Further, in some embodiments, the method 1100 further may include a step 1102 of analyzing, using the processing device 704, the pre-diagnosis and/or diagnosis report data comprising the encrypted data. Further, in some embodiments, the method 1100 further may include a step 1104 of generating, using the processing device 704, a compliance parameter based on the analyzing.

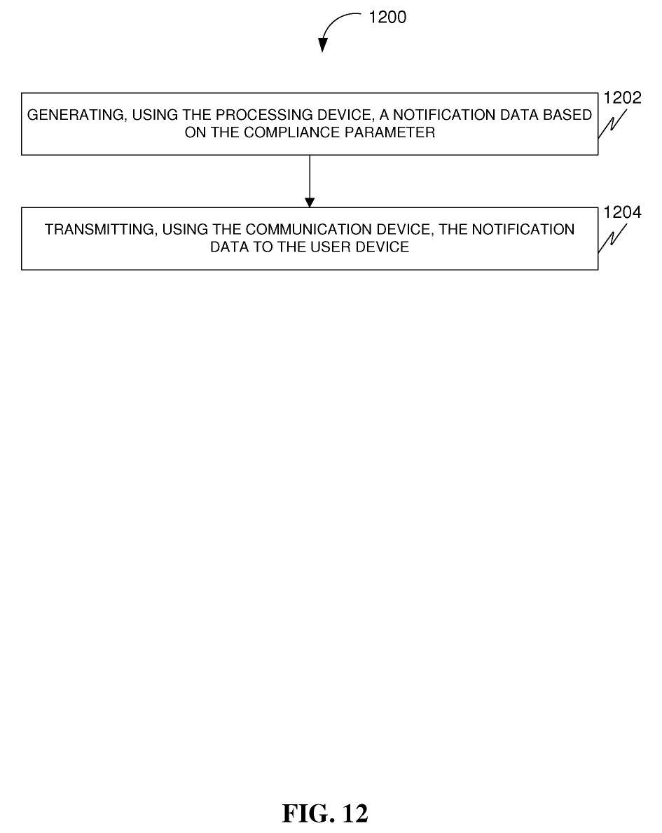

FIG. 12 illustrates a flowchart of a method 1200 of facilitating AI-driven refinement of mental health diagnostics and personalized treatment including generating, using the processing device 704, a notification data, in accordance with some embodiments.

Further, in some embodiments, the method 1200 further may include a step 1202 of generating, using the processing device 704, a notification data based on the compliance parameter. Further, in some embodiments, the method 1200 further may include a step 1204 of transmitting, using the communication device 702, the notification data to the user device.

In some embodiments, the therapy data includes one or more of an audio data, a video data, a text data and an image data.

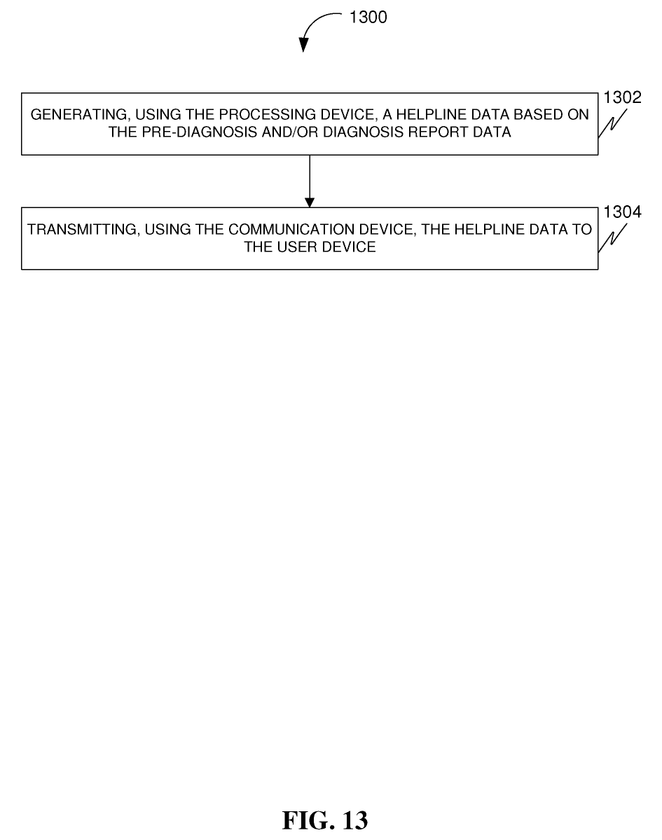

FIG. 13 illustrates a flowchart of a method 1300 of facilitating AI-driven refinement of mental health diagnostics and personalized treatment including generating, using the processing device 704, a helpline data, in accordance with some embodiments.

Further, in some embodiments, the method 1300, further may include a step 1302 of generating, using the processing device 704, a helpline data based on the pre-diagnosis and/or diagnosis report data. Further, in some embodiments, the method 1300, further may include a step 1304 of transmitting, using the communication device 702, the helpline data to the user device.

In some embodiments, the pre-diagnosis and/or diagnosis report data includes a result data corresponding to a medical condition associated with the user. Further, the result data includes one or more of a description of the medical condition and a symptom related to the user.

In some embodiments, the prompt data corresponds to a group discussion. Further, the group discussion includes at least two users comprising the user.

In some embodiments, the user device includes a first user device associated with a first user and a second user device associated with a second user. Further, the prompt data may be received from a second user device. Further, the prompt data may be transmitted to the first user device.

In some embodiments, the therapy data includes indication of a therapy appointment of the user with a psychologist.

In some embodiments, the prompt data includes an interview data representing an interview. Further, the user device includes a presentation device which may be configured to present the interview data. Further, the user device further includes one or more of an input device and a user communication device. Further, the input device may be configured to receive a response data from the user based on the presentation of the interview data. Further, the user communication device may be configured to receive the response data from an external device which may be configured to generate the response data provided by the user. Further, the mental health data includes the response data.

In some embodiments, the mental health data includes a pre-existing health data of the user.

In some embodiments, the mental health data includes a user profile data associated with the user. Further, the updating of the diagnostic schema data may be based on the user profile data.

In some embodiments, the method 300 may further include updation of the user profile data based on one or more of the mental health data, the diagnostic schema data and the pre-diagnosis and/or diagnosis report data.

In some embodiments, the method 300 may further include encrypting a part of pre-existing health data to obtain an encrypted health data. Further, the pre-existing health data includes the encrypted health data in place of the part of pre-existing health data. Further, the encrypting may be in compliance with one or more healthcare regulations.

In some embodiments, the method 300 may further include encrypting a part of pre-diagnosis and/or diagnosis data to obtain an encrypted diagnosis data. Further, the pre-diagnosis and/or diagnosis data includes the encrypted diagnosis data in place of the part of pre-diagnosis and/or diagnosis data. Further, the encrypting may be in compliance with one or more healthcare regulations.

In some embodiments, the diagnostic schema data includes a diagnostic ontology data. Further, the diagnostic ontology data includes one or more diagnostic domain data. Further, the diagnostic domain data corresponds to the mental health data.

In some embodiments, the user device includes a presentation device and an input device. Further, the presentation device may be configured to display the prompt data to a user associated with the user device and the input device may be configured to capture the mental health data based on a response of the user to the prompt data.

In some embodiments, the prompt data includes a descriptive data representing one or more of an image and a video. Further, the user device includes a presentation device which may be configured to present the descriptive data. Further, an involuntary motion may be performable by the user based on the presentation of the descriptive data. Further, the user device further includes a motion sensor which may be configured to detect the involuntary motion. Further, the user device further includes a user processing device which may be configured to generate a motion data based on the detection. Further, the mental health data includes the motion data.

In some embodiments, the prompt data includes an audio data. Further, the user device includes a presentation device which may be configured to present the audio data. Further, an involuntary action may be performable by the user based on the presentation of the audio data. Further, the user device further includes an audio sensor which may be configured to detect the involuntary action. Further, the user device further includes a user processing device which may be configured to generate a sensor data based on the detection. Further, the mental health data includes the sensor data.

In some embodiments, the prompt data includes a haptic data. Further, the user device includes a presentation device which may be configured to present the haptic data. Further, an involuntary action may be performable by the user based on the presentation of the haptic data. Further, the user device further includes a haptics sensor which may be configured to detect the involuntary action. Further, the user device further includes a user processing device which may be configured to generate a haptic sensor data based on the detection. Further, the mental health data includes the haptic sensor data.

In some embodiments, the prompt data includes a descriptive data representing one or more of an image and a video. Further, the user device includes a presentation device which may be configured to present the descriptive data. Further, a voluntary motion may be performable by the user based on the presentation of the descriptive data. Further, the user device further includes a motion sensor which may be configured to detect the voluntary motion. Further, the user device further includes a user processing device which may be configured to generate a motion data based on the detection. Further, the mental health data includes the motion data.

In some embodiments, the prompt data includes an audio data. Further, the user device includes a presentation device which may be configured to present the audio data. Further, a voluntary action may be performable by the user based on the presentation of the audio data. Further, the user device further includes an audio sensor which may be configured to detect the voluntary action. Further, the user device further includes a user processing device which may be configured to generate a sensor data based on the detection. Further, the mental health data includes the sensor data.

In some embodiments, the prompt data includes a haptic data. Further, the user device includes a presentation device which may be configured to present the haptic data. Further, a voluntary action may be performable by the user based on the presentation of the haptic data. Further, the user device further includes a haptics sensor which may be configured to detect the voluntary action. Further, the user device further includes a user processing device which may be configured to generate a haptic sensor data based on the detection. Further, the mental health data includes the haptic sensor data.

In some embodiments, the user device may be operated by one or more of a family member, a doctor, a nurse and a friend on behalf of the user.

In some embodiments, the mental health data includes one or more of a text data, a voice data, an image data, a haptic data and a biometric data.

In some embodiments, the plurality of the medical domain indicators represents one or more of a neurological domain, a psychiatry domain and a neuropsychology domain.

In some embodiments, the method 300 may further include storing, using a storage device, the encrypted data.

In some embodiments, the user device may be one or more of a smartphone, a laptop computer, a tablet computer, a wearable computer and a desktop computer.

In some embodiments, the provisioning of the service associated with mental health may be related to a field of digital health.

In some embodiments, the mental health data includes an age data representing an age of a user associated with the mental health data.

In some embodiments, the diagnostic schema data represents a framework for diagnosing a medical condition. Further, the medical condition corresponds to the mental health data.

In some embodiments, the lifestyle data includes an exercise data representing a physical activity status of the user.

In some embodiments, the lifestyle data includes a sleep data representing a sleeping pattern of the user.

In some embodiments, the lifestyle data includes a diet data representing an eating habit of the user.

In some embodiments, the mental health data includes a gender data representing a gender of the user.

In some embodiments, the mental health data includes an occupation data representing a profession of the user.

In some embodiments, the mental health data includes a relationship data representing a status of relationship of the user.

In some embodiments, the user device includes a user processing device which may be configured to track a usage of a digital platform by the user and further generate a digital usage data. Further, the pre-diagnosis and/or diagnosis report data may be generated based on the digital usage data.

In some embodiments, the mental health data includes a personality data representing qualities of the user.

In some embodiments, the generation of therapy data may be further based on an identification of similarity between symptoms data related to the user and characteristics of a medical condition.

In some embodiments, the method 500 may further include encrypting a part of the therapy data to obtain an encrypted therapy data. Further, the therapy data includes the encrypted therapy data in place of the part of the therapy data.

In some embodiments, the method 300 may further include encrypting a part of the mental health data to obtain an encrypted mental health data. Further, the mental health data includes the encrypted mental health data in place of the part of the mental health data. Further, the encrypting may be in compliance with one or more healthcare regulations.

In some embodiments, the prompt data includes a standard collection of questions.

In some embodiments, one or more of the mental health data and a user feedback data provided by the user enhances the accuracy of one or more of the diagnostic schema data and the pre-diagnosis and/or diagnosis report data.

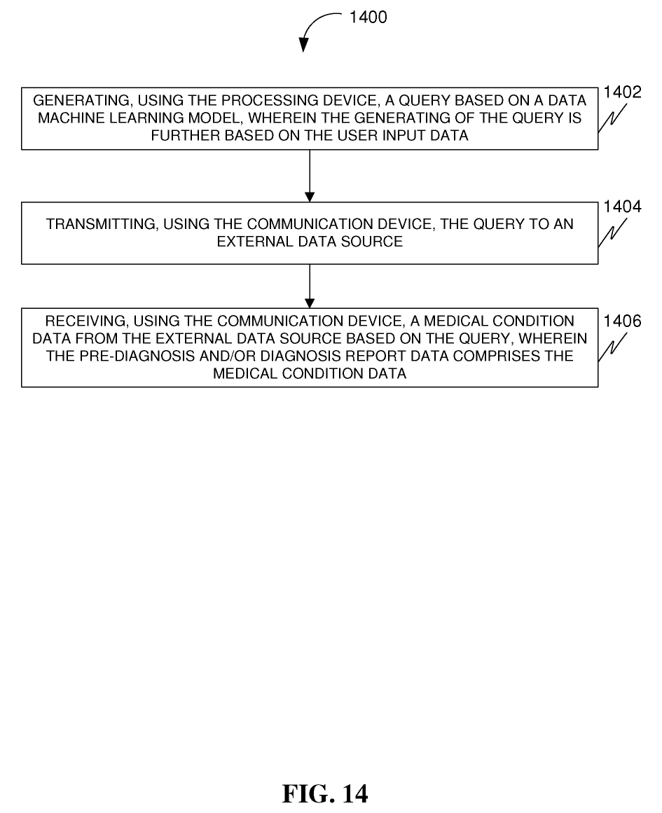

FIG. 14 illustrates a flowchart of a method 1400 of facilitating AI-driven refinement of mental health diagnostics and personalized treatment including receiving, using the communication device 702, a medical condition data from the external data source, in accordance with some embodiments.

Further, in some embodiments, the mental health data may include a user input data associated with a medical condition. Further, the method 1400 further may include a step 1402 of generating, using the processing device 704, a query based on a data machine learning model. Further, the generating of the query may be further based on the user input data. Further, the method 1400 further may include a step 1404 of transmitting, using the communication device 702, the query to an external data source. Further, the method 1400 further may include a step 1406 of receiving, using the communication device 702, a medical condition data from the external data source based on the query. Further, the pre-diagnosis and/or diagnosis report data includes the medical condition data.

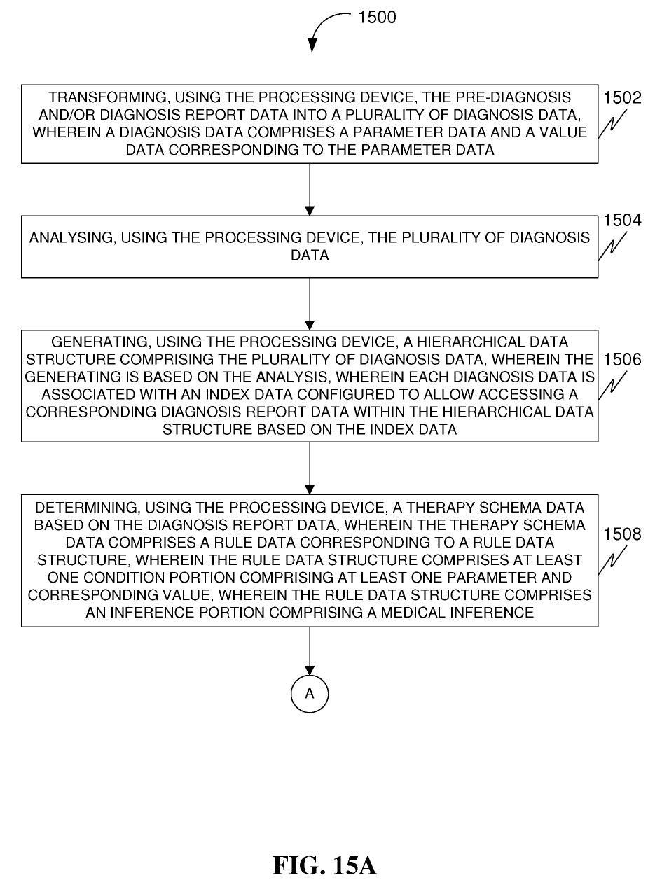

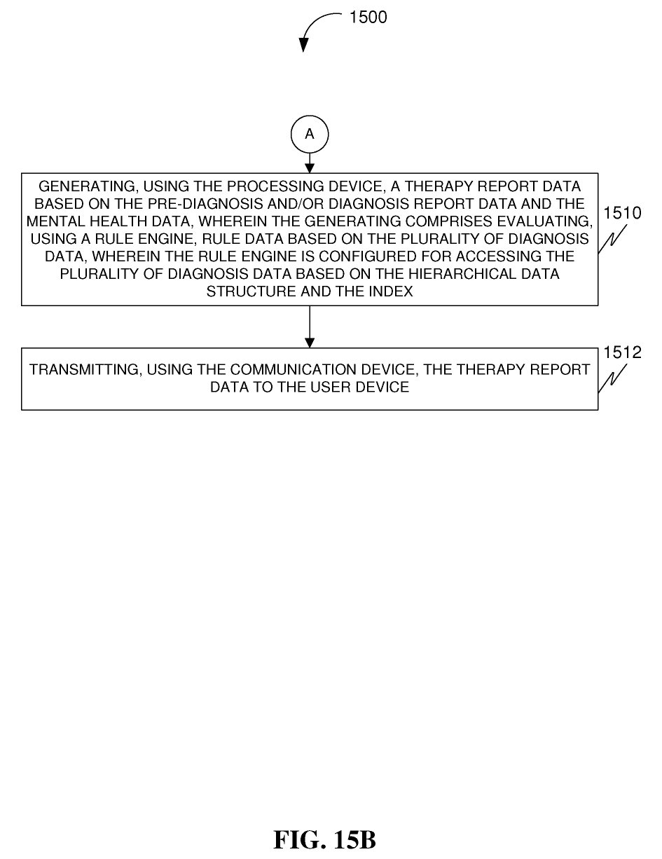

FIG. 15A and FIG. 15B illustrate a flowchart of a method 1500 of facilitating AI-driven refinement of mental health diagnostics and personalized treatment including generating, using the processing device 704, a therapy report data, in accordance with some embodiments.

Further, in some embodiments, the method 1500 further may include a step 1502 of transforming, using the processing device 704, the pre-diagnosis and/or diagnosis report data into two or more diagnosis data. Further, a diagnosis data includes a parameter data and a value data corresponding to the parameter data. Further, in some embodiments, the method 1500 further may include a step 1504 of analyzing, using the processing device 704, the two or more diagnosis data. Further, in some embodiments, the method 1500 further may include a step 1506 of generating, using the processing device 704, a hierarchical data structure comprising the two or more diagnosis data. Further, the generating may be based on the analysis. Further, each diagnosis data may be associated with an index data which may be configured to allow accessing a corresponding pre-diagnosis and/or diagnosis report data within the hierarchical data structure based on the index data. Further, in some embodiments, the method 1500 further may include a step 1508 of determining, using the processing device 704, a therapy schema data based on the pre-diagnosis and/or diagnosis report data. Further, the therapy schema data includes a rule data corresponding to a rule data structure. Further, the rule data structure includes one or more condition portions comprising one or more parameters and corresponding value. Further, the rule data structure includes an inference portion comprising a medical inference. Further, in some embodiments, the method 1500 further may include a step 1510 of generating, using the processing device 704, a therapy report data based on the pre-diagnosis and/or diagnosis report data and the mental health data. Further, the generating includes evaluating, using a rule engine, rule data based on the two or more diagnosis data. Further, the rule engine may be configured for accessing the two or more diagnosis data based on the hierarchical data structure and the index. Further, in some embodiments, the method 1500 further may include a step 1512 of transmitting, using the communication device 702, the therapy report data to the user device.

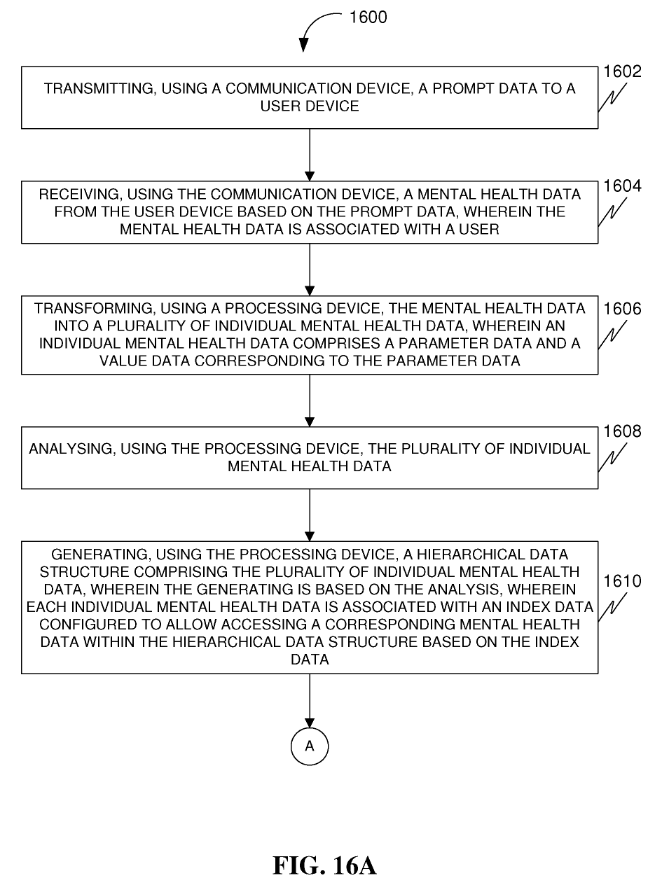

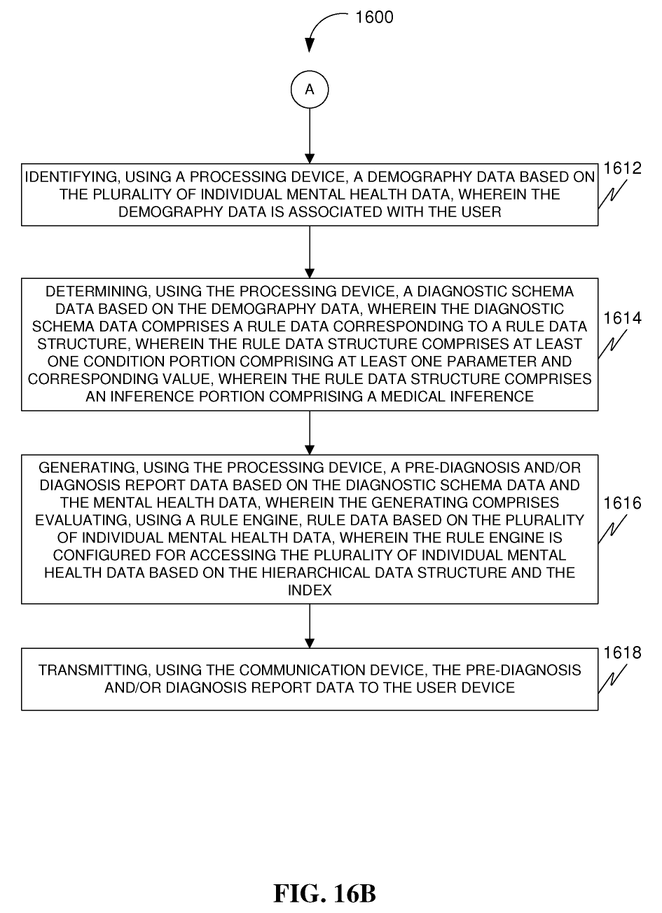

FIG. 16A and FIG. 16B illustrate a flowchart of a method 1600 of facilitating AI-driven refinement of mental health diagnostics and personalized treatment, in accordance with some embodiments.

Accordingly, the method 1600 may include a step 1602 of transmitting, using a communication device 702, a prompt data to a user device. Further, the method 1600 may include a step 1604 of receiving, using the communication device 702, a mental health data from the user device based on the prompt data. Further, the mental health data may be associated with a user. Further, the method 1600 may include a step 1606 of transforming, using a processing device 704, the mental health data into two or more individual mental health data. Further, an individual mental health data includes a parameter data and a value data corresponding to the parameter data. Further, the method 1600 may include a step 1608 of analyzing, using the processing device 704, the two or more individual mental health data. Further, the method 1600 may include a step 1610 of generating, using the processing device 704, a hierarchical data structure comprising the two or more individual mental health data. Further, the generating may be based on the analysis. Further, each individual mental health data may be associated with an index data which may be configured to allow accessing a corresponding mental health data within the hierarchical data structure based on the index data. Further, the method 1600 may include a step 1612 of identifying, using a processing device 704, a demography data based on the two or more individual mental health data. Further, the demography data may be associated with the user. Further, the method 1600 may include a step 1614 of determining, using the processing device 704, a diagnostic schema data based on the demography data. Further, the diagnostic schema data includes a rule data corresponding to a rule data structure. Further, the rule data structure includes one or more condition portions comprising one or more parameters and corresponding value. Further, the rule data structure includes an inference portion comprising a medical inference. Further, the method 1600 may include a step 1616 of generating, using the processing device 704, a pre-diagnosis and/or diagnosis report data based on the diagnostic schema data and the mental health data. Further, the generating includes evaluating, using a rule engine, rule data based on the two or more individual mental health data. Further, the rule engine may be configured for accessing the two or more individual mental health data based on the hierarchical data structure and the index. Further, the method 1600 may include a step 1618 of transmitting, using the communication device 702, the pre-diagnosis and/or diagnosis report data to the user device.

Although the invention has been explained in relation to its preferred embodiment, it is to be understood that many other possible modifications and variations can be made without departing from the spirit and scope of the invention as hereinafter claimed.

1. A method of facilitating AI-driven refinement of mental health diagnostics and personalized treatment, the method comprising:
transmitting, using a communication device, a prompt data to a user device;
receiving, using the communication device, a mental health data from the user device based on the prompt data, wherein the mental health data is associated with a user;
identifying, using a processing device, a demography data based on the mental health data, wherein the demography data is associated with the user;
determining, using the processing device, a diagnostic schema data based on the demography data;
generating, using the processing device, a pre-diagnosis and/or diagnosis report data based on the diagnostic schema data and the mental health data; and
transmitting, using the communication device, the pre-diagnosis and/or diagnosis report data to the user device.

2. The method of claim 1 further comprising:
generating, using the processing device, a query corresponding to the diagnostic schema data based on a diagnostic machine learning model, wherein the generating of the query is further based on at least one of the mental health data and the pre-diagnosis and/or diagnosis report data;
transmitting, using the communication device, the query to an external data source, wherein the external data source comprises a diagnostic data associated with the diagnostic schema data;
receiving, using the communication device, the diagnostic data based on the query; and
performing, using the processing device, an updation of the diagnostic schema data based on the diagnostic data.

3. The method of claim 1, further comprising encrypting a part of the pre-diagnosis and/or diagnosis report data to obtain an encrypted data, wherein the pre-diagnosis and/or diagnosis report data comprises the encrypted data in place of the part of the pre-diagnosis and/or diagnosis report data.

4. The method of claim 1, wherein the prompt data represents an activity performable by the user, wherein the mental health data is generated based on a performance of the activity, wherein the activity comprises at least one of an emotion expression task, a speech task and a focusing task, wherein the user device comprises at least one sensor configured to generate the mental health data based on the activity, wherein the at least one sensor comprises each of a camera and a microphone.

5. The method of claim 1, wherein the pre-diagnosis and/or diagnosis report data comprises a pre-diagnosis and/or diagnosis data, wherein the pre-diagnosis and/or diagnosis data is transmitted to a clinician device associated with a clinician.

6. The method of claim 1 further comprising determining, using the processing device, a plurality of medical domain indicators corresponding to the mental health data based on a domain machine learning model, wherein the determining comprises performing a first analysis of the mental health data.

7. The method of claim 1, further comprising:
generating, using the processing device, a therapy data based on a therapy machine learning model comprising a multi-modal deep learning module, wherein at least one of the pre-diagnosis and/or diagnosis report data corresponds to a plurality of modalities, wherein the therapy data indicates a therapy, wherein the generating of the therapy data comprises performing a second analysis of the pre-diagnosis and/or diagnosis report data; and
transmitting, using the communication device, the therapy data to the user device.

8. The method of claim 7, further comprises:
generating, using the processing device, a user feedback form data based on at least one of the mental health data, the diagnostic schema data, the pre-diagnosis and/or diagnosis report data and the therapy data;
transmitting, using the communication device, the user feedback form data to the user device;
receiving, using the communication device, a user feedback data based on the user feedback form data; and
determining, using the processing device, an alternative therapy data based on the user feedback data, wherein the alternative therapy data indicates an improved therapy on the user; and
transmitting, using the communication device, the alternative therapy data to the user device.

9. The method of claim 1, wherein the mental health data comprises a lifestyle data representing a manner of living of the user.

10. The method of claim 1 further comprises generating, using the processing device, the prompt data, wherein the mental health data comprises a first mental health data and a second mental health data, wherein the prompt data comprises a first prompt data and a second prompt data, wherein the first mental health data is received in response to the first prompt data and the second mental health data is received in response to the second prompt data, wherein the generating of the second prompt data is based on the first mental health data.

11. A system for facilitating AI-driven refinement of mental health diagnostics and personalized treatment, the system comprising:
a communication device configured for:
transmitting a prompt data to a user device;
receiving a mental health data from the user device based on the prompt data, wherein the mental health data is associated with a user;
transmitting a pre-diagnosis and/or diagnosis report data to the user device; and
a processing device configured for:
identifying a demography data associated with the user based on the mental health data;
identifying a diagnostic schema data based on the demography data; and
generating the pre-diagnosis and/or diagnosis report data based on the diagnostic schema data.

12. The system of claim 11, wherein the processing device is further configured for:
generating a query corresponding to the diagnostic schema data based on a diagnostic machine learning model, wherein the generating of the query is further based on at least one of the mental health data and the pre-diagnosis and/or diagnosis report data; and
performing an updation of the diagnostic schema data based on the diagnostic data, wherein the communication device is further configured for:
transmitting the query to an external data source, wherein the external source comprises the diagnostic data related to the diagnostic schema data; and
receiving the diagnostic data from the external data source based on the query.

13. The system of claim 11, wherein the processing device is further configured for encrypting a part of the pre-diagnosis and/or diagnosis report data to obtain an encrypted data, wherein the pre-diagnosis and/or diagnosis report data comprises the encrypted data in place of the part of the pre-diagnosis and/or diagnosis report data.

14. The system of claim 11, wherein the prompt data represents an activity, wherein the activity is performable by the user, wherein the mental health data is generated based on a performance of the activity, wherein the user device comprises at least one sensor configured to generate the mental health data based on the activity, wherein the at least one sensor comprises each of a camera and a microphone.

15. The system of claim 11, wherein the pre-diagnosis and/or diagnosis report data comprises a pre-diagnosis and/or diagnosis data, wherein the communication device is further configured for transmitting the pre-diagnosis and/or diagnosis data to a clinician device associated with a clinician.

16. The system of claim 11, wherein the processing device is further configured for determining a plurality of medical domain indicators corresponding to the mental health data based on a domain machine learning model, wherein the determining comprises performing a first analysis of the mental health data.

17. The system of claim 11, wherein the processing device is further configured for generating a therapy data based on a therapy machine learning model comprising a multi-modal deep learning module, wherein at least one of the pre-diagnosis and/or diagnosis report data corresponds to a plurality of modalities, wherein the therapy data indicates a therapy, wherein the generating comprises performing a second analysis of the pre-diagnosis and/or diagnosis report data, wherein the communication device is further configured for transmitting the therapy data to the user device.

18. The system of claim 17, wherein the processing device is further configured for:
generating a user feedback form data based on at least one of the mental health data, the diagnostic schema data, the pre-diagnosis and/or diagnosis report data and the therapy data; and
determining an alternative therapy data based on a user feedback data, wherein the alternative therapy data indicates an enhanced, personalized therapy on the user, wherein the communication device is further configured for:
transmitting the user feedback form data to the user device;
receiving a user feedback data based on the user feedback form data; and
transmitting the alternative therapy data to the user device.

19. The system of claim 11, wherein the mental health data comprises a lifestyle data representing a manner of living of the user.

20. The system of claim 11, wherein the processing device is further configured for generating the prompt data, wherein the mental health data comprises a first mental health data and a second mental health data, wherein the prompt data comprises a first prompt data and a second prompt data, wherein the first mental health data is received in response to the first prompt data and the second mental health data is received in response to the second prompt data, wherein the generating of the second prompt data is based on the first mental health data.
